(19) United States
(12) Patent Application Publication
(10) Pub. No.: US 2026/0119516 A1
(43) Pub. Date: Apr. 30, 2026

(54) TECHNIQUES FOR CONSTRAINT-DRIVEN QUERY ROUTING OVER DISPARATE DATA STORES

(71) Applicant: Oracle International Corporation, Redwood Shores, CA (US)

(72) Inventor: Raman Grover, Palo Alto, CA (US)

(73) Assignee: Oracle International Corporation, Redwood Shores, CA (US)

(21) Appl. No.: 19/368,525

(22) Filed: Oct. 24, 2025

Related U.S. Application Data
(60) Provisional application No. 63/711,943, filed on Oct. 25, 2024.

(51) Int. Cl. G06F 16/248; G06F 16/21; G06F 16/2455

(52) U.S. Cl. CPC G06F 16/248; G06F 16/211; G06F 16/24553

(57) ABSTRACT

Techniques are disclosed for constraint-driven query routing in heterogeneous data environments with disparate data stores. In one aspect, a method includes receiving a query in a first programming language and associated with one or more constraints. The constraints can include freshness, feasibility, divergence, and/or execution time. An intent of the query is identified, and a dry run of the query is performed to evaluate whether a data store satisfies the constraints. An optimal data store is selected based on the dry run. A query result is generated by determining whether the query in the first programming language can be executed on the optimal data store. The query is executed on the optimal data store if the first programming language is executable on the optimal data store. Otherwise, the query is converted to a second query in a second programming that can be executed on the optimal data store.

### CROSS-REFERENCE TO RELATED APPLICATION

The present application is a non-provisional application of and claims the benefit and priority under 55 U.S.C. 119 (e) of U.S. Application 63/711,943, filed on Oct. 25, 2024, the disclosure of which is incorporated herein by reference in its entirety for all purposes.

### FIELD

The present disclosure relates generally to data systems, and more particularly, to techniques for improved data processing and query routing in distributed data storage systems.

### BACKGROUND

Heterogeneous and disparate data stores can make computing and querying data more flexible and efficient. Applications that interface with data storage systems can better query data that suit their needs, rather than being limited to a particular type of data query or store. The data storage system can also scale better to better optimize for different workloads.

In recent years, there has been a significant rise in capabilities of data storage systems. In particular, improvements in natural language processing (NLP) have increased the abilities of data storage systems to store semantic concepts of data stored with the storage system. Managing and processing data across various components, particularly for data storage systems with disparate data stores, data models, or the like can be difficult to maintain.

The improvement of data storage systems represents a significant advancement in making data storage systems more accessible and accurate. By improving capabilities of data storage systems, these systems can improve access to information across applications. This disclosure presents techniques related to improved data processing techniques in distributed data storage systems.

### BRIEF SUMMARY

Data processing techniques are disclosed herein (e.g., computer-implemented methods, systems, non-transitory computer-readable media storing code or instructions executable by one or more processors) for constraint driven query routing for data storage systems with disparate data stores, enabling improved query processing and data retrieval in data systems.

In some embodiments, a computer-implemented includes receiving, at a data storage system comprising a plurality of data stores, a query, wherein: the query is associated with one or more constraints, the one or more constraints comprise (i) a freshness constraint, (ii) a feasibility constraint, (iii) an execution time constraint, or (iv) any combination thereof, and the query is in a first programming language; identifying, from a set of intents, an intent of the query based on one or more key terms within the query; performing a dry run of the query on at least a subset of the plurality of data stores to evaluate, for each data store of at least the subset, whether the data store satisfies the one or more constraints based on the intent and data store metadata, wherein the data store metadata comprises data store information associated with each data store of at least the subset; selecting, based on the dry run, an optimal data store from at least the subset of the plurality of data stores; generating a query result for the query, wherein generating the query result comprises: determining whether the query in the first programming language can be executed on the optimal data store, in response to determining the query in the first programming language can be executed on the optimal data store, executing the query on the optimal data store to obtain the query result, and in response to determining the query in the first programming language cannot be executed on the optimal data store: converting the query to a second query in a second programming language that can be executed on the optimal data store, and executing the second query on the optimal data store to obtain the query result; and providing the query result.

In some embodiments, the data store information is associated with (i) a data model of the data store, (ii) a schema of the data store, (iii) a set of features of the data store, or (iv) any combination thereof.

In some embodiments, the set of intents comprises (i) point query, (ii) filter query, (iii) join query, (iv) aggregate query, (v) subquery, or (vi) any combination thereof.

In some embodiments, the one or more constraints comprise the freshness constraint, and wherein performing the dry run comprises, for each data store of at least the subset: computing a watermark indicating a freshness of data within the data store based on the data store metadata; and determining whether the data store satisfies the freshness constraint based on a comparison of the watermark and an expected freshness indicated by the freshness constraint.

In some embodiments, the one or more constraints comprise the feasibility constraint; performing the dry run comprises, for each data store of at least the subset, determining whether the query can be executed on the data store based on the intent and one or more features of the data store; and the one or more features are determined based on the data store metadata.

In some embodiments, performing the dry run comprises, for each data store of at least the subset: determining a constraint satisfaction score for each constraint of the one or more constraints based on the intent and the data store metadata; computing a weighted sum of the constraint satisfaction scores; determining whether the weighted sum matches or exceeds a constraint satisfaction threshold; in response to determining the weighted sum matches or exceeds the constraint satisfaction threshold, determining the data store satisfies the one or more constraints; and in response to determining the weighted sum does not match or exceed the constraint satisfaction threshold, determining the data store does not satisfy the one or more constraints.

In some embodiments, the one or more constraints are expressed in a constraint definition language (CDL), and wherein the expression of the one or more constraints in the CDL indicates a logical precedence of the one or more constraints.

In some embodiments, the computer-implemented method further includes: collecting execution metadata associated with the execution of the query or the execution of the second query, the execution metadata comprising at least one of (i) query response time, (ii) freshness level, (iii) feasibility status, (iv) user satisfaction, or (v) any combination thereof; receiving a subsequent query associated with at least one of the one or more constraints; and selecting the optimal data store based on the execution metadata and historical query execution data.

In some embodiments, converting the query comprises: providing, to a generative model, a prompt comprising the query and one or more instructions to translate the query from the first programming language to the second programming language; and receiving, from the generative model, the second query corresponding to the second programming language, wherein the generative model generates the second query based on the prompt, wherein the second programming language is a programming language that can be executed on the optimal data store.

In some embodiments, selecting the optimal data store comprises applying a machine learning decision model trained on historical query execution patterns.

Some embodiments include a system that includes one or more processors; and one or more computer-readable media storing instructions which, when executed by the one or more processors, cause the system to perform part or all of the operations and/or methods disclosed herein.

Some embodiments include one or more non-transitory computer-readable media storing instructions which, when executed by one or more processors, cause a system to perform part or all of the operations and/or methods disclosed herein.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

FIG. 1 is an example of an architecture for a computing environment for a clinical digital assistant in accordance with various embodiments.

FIG. 2 is a block diagram of a digital assistant runtime flow with components and

interfaces into a semantic index, in accordance with various embodiments.

FIG. 3 is a simplified block diagram of a computing environment of a disparate data storage system, in accordance with various embodiments.

FIG. 4 is an example of an architecture for a computing environment for a data storage system implemented with an ingestion flow for disparate data stores, in accordance with various embodiments.

FIG. 5 is a simplified block diagram of a watermark generation and evaluation within a data ingestion flow, in accordance with various embodiments.

FIG. 6 is a block diagram of a data storage system implementing a constraint-driven query routing with heterogeneous data stores, in accordance with various embodiments.

FIG. 7 is a block diagram illustrating data flow for constraint-based query routing in a data storage system, in accordance with various embodiments.

FIG. 8 is a flowchart of a process constraint-driven query routing in data systems with disparate data stores, in accordance with various embodiments.

FIG. 9 is a block diagram illustrating one pattern for implementing a cloud infrastructure as a service system, in accordance with various embodiments.

FIG. 10 is a block diagram illustrating another pattern for implementing a cloud infrastructure as a service system, in accordance with various embodiments.

FIG. 11 is a block diagram illustrating another pattern for implementing a cloud infrastructure as a service system, in accordance with various embodiments.

FIG. 12 is a block diagram illustrating another pattern for implementing a cloud infrastructure as a service system, in accordance with various embodiments.

FIG. 13 is a block diagram illustrating an example computer system, in accordance with various embodiments.

### DETAILED DESCRIPTION

In the following description, for the purposes of explanation, specific details are set forth in order to provide a thorough understanding of certain embodiments. However, it will be apparent that various embodiments may be practiced without these specific details. The figures and description are not intended to be restrictive. The word “exemplary” is used herein to mean “serving as an example, instance, or illustration.” Any embodiment or design described herein as “exemplary” is not necessarily to be construed as preferred or advantageous over other embodiments or designs.

### INTRODUCTION

In recent years, the amount of data powering various industries and their systems has been increasing exponentially. Organizations and businesses store and consume data across various types of data stores (e.g., relational databases, non-relational databases, object stores, key-value stores, file storage, etc.). These data stores power information systems across multiple industries, for instance, consumer tech (e.g., orders, cancellations, refunds), supply chain (e.g., raw materials, stocks, vendors), healthcare (e.g., medical records), finance (e.g., financial business metrics), customer support, search engines, and much more. Data that powers these industries can come from a variety of different sources and it is imperative for modern data-driven organizations to maintain consistent and reliable data to provide accurate representations of data to users.

With the rise of natural language (NL) processing and artificial intelligence capabilities, storing and providing data in ways that maintain semantic coherence and meaning can improve user queries and interactions with data storage systems. It is vastly more efficient for non-technical users (e.g., business leaders, doctors, or other users of the data) directly interact with analytics tables via natural language (NL) queries that abstract away underlying query language and/or data structures of a data storage system. Further, for data storage systems with multiple sources of data reflected within the storage systems, querying the system with a single unified structure can make accessing data more efficient and reduce user burden. By providing unified query and storage structures, even technical users with strong understandings of one type of data storage but lacking knowledge in other types of data storage can better query a data storage system based on types of data storage and querying implementations they are comfortable with.

Implementing a Semantic Object Model in a data environment with disparate and/or distributed data stores can be a powerful tool for unifying data across disparate and/or distributed data stores while providing efficient access to data. Unlike other data models, which often only define objects by structure, a Semantic Object Model can define objects by their semantic meaning and relationships. Objects are represented as concepts associated with various attributes and relationships that can be leveraged to determine semantics and meaning. For example, in a healthcare environment, a patient can be represented as a concept, and semantic objects corresponding to a patient concept can include various attributes that can describe the patient such as name, address, phone number, and the like. In some implementations, semantic objects can include self-describing metadata and/or linked actions for custom operations.

In heterogeneous data environments, which can include multiple data stores with varying data types, data models, data sources, querying functionality support, and storage technology, providing a unified method of querying data can be an important way to abstract implementations of data stores within the data system. Without a unified method of querying, queries to the data storage system may be targeted to a singular data store or type of data store. It would be more beneficial for queries to be processed and executed agnostic to specific data stores by routing queries to a data store within the heterogeneous data system that can optimally respond to the query. Providing such unified methods across varying data models, schemas, and querying implementations in the heterogeneous data environment, however, can be challenging.

Moreover, many heterogeneous data systems maintain consistency across data stores within the system and/or with an external source data system. To maintain consistency across data stores, various data ingestion flows can be implemented that perform data replication and propagate updates from the source system to the target system. Target systems are susceptible to lag and divergence, however, due to limitations of data replication and propagation across data systems and between data stores. Lag can refer to the delay (e.g., number of time units) between a data update occurring in one node (e.g., a source data store) and being propagated to another node (e.g., a target data store). Factors including but not limited to data processing delays, throttling, and network latency can impact lag in a distributed data storage system. In some instances, lag can occur due to infrastructure issues caused by constrained system resources that may not be sufficient in handling an increased load experienced by the system. In such instances, lag may correct over time when load is reduced and/or additional resources are added to the system to handle the load.

Divergence can refer to a difference in state between a source system and a target system. In systems with no divergence, all target and source data stores may store the same versions and/or values of data, while in systems with high divergence, data stored in target data stores and source data stores may have significant differences. Unlike lag, however, disagreements between data in target and source data systems caused by divergence may perpetuate until a cause for the divergence is directly addressed. For example, divergence may be caused by a software bug or system misconfiguration that causes errors in changes to data values or ingestion of new data. Software bugs or system misconfigurations often do not correct over time and are instead typically addressed by explicit correction of the issue. Accordingly, routing queries with an awareness of data correctness and data freshness, which can refer to how recent and accurate the data is, is important in providing accurate data access. Because certain data stores can store different data when experiencing lag and/or divergence, routing queries without considering freshness can be detrimental or even damaging for end users (e.g., a doctor requesting time-sensitive treatment information).

Furthermore, ensuring that queries can be feasibly executed on a data store based on an intent of the query is important to ensure that a query can perform the intended operations. For example, certain data stores may be unable to handle certain types of queries and a system should avoid routing queries to data stores that cannot handle the queries. Without consideration of the feasibility of a data store to execute a query intent, the data system may experience a large number of execution failures that can impede data access for users and other entities. Feasibility of a query may be distinct from whether the query in the particular programming language (e.g., programming query languages such as SQL, Vector DSL, Query DSL, etc.) and/or syntax is executable on data store. For example, many data stores (e.g., relational databases, vector databases, etc.) support identifier-based lookups and feasibility of a data store may be determined based on whether a data store can support an intent of performing an identifier-based lookup, rather than whether the data store supports the programming language of the query. As used herein, programming language can refer to a database query language (e.g., SQL, PQL, SPARQL, and the like), API query language (e.g., GraphQL, REST, etc.), developer programming language (e.g., Python, C++, Java, Ruby, etc.), and the like. For a data system to consider the feasibility of a query based on the intent, processing and routing queries in a manner agnostic to programming language is important. This, however, is a challenge that is not addressed by conventional techniques for query routing.

Conventional techniques for query routing are often inadequate for routing queries in heterogeneous data system, however. Implementations of query routing typically fail to consider multi-dimensional constraints such as feasibility, divergence, and freshness, which can lead to suboptimal query performance, inconsistent results, and unnecessary load on certain data stores within the system. For example, conventional query optimizers such as those implemented in relational databases perform cost-based optimization by estimating computational resources required to execute a query. However, cost-based optimization is limited in heterogeneous environments, where some data stores in the environment may not be able to execute certain queries and thus fail to satisfy feasibility constraints. As another example, heuristic-based routing approaches lack adaptability and may be unable to consider real-time factors such as data freshness, and other query constraints. As a result, conventional query routing techniques struggle to meet the data access and retrieval requirements of users and other entities querying data.

To overcome these challenges and others, a technical solution involving data processing techniques for constraint-driven query routing in heterogeneous data environments has been developed. A query received at a data storage system can be associated with constraint(s) including but not limited to freshness, feasibility, divergence and execution time. Optionally, the query constraints can be expressed in a constraint definition language (CDL) that indicates a logical precedence of the constraints. An intent of the query is identified based on attributes of the query. Attributes of the query can include key terms of the query (e.g., keywords, operators, clauses, expressions, etc.) that can be used to uniquely identify the intent of the query, Based on the identified intent, a dry run evaluation is performed on at least a subset of the data stores within the data storage system to determine an optimal data store that best satisfies the constraint(s). The dry run utilizes metadata information associated with each data store to determine constraint satisfaction. If the query is in a programming language that is executable on the optimal data store, the query is executed to retrieve a query result. In some instances, the query may be provided in a programming language that is not executable on the optimal data store. For such instances, the query is dynamically translated into a programming language supported by the optimal data store and subsequently executed on the optimal data store to retrieve the query result. The query result can then be provided to a user or other entity.

In one exemplary embodiment, a computer-implemented method is provided that includes receiving, at a data storage system comprising a plurality of data stores, a query, wherein: the query is associated with one or more constraints, the one or more constraints comprise (i) a freshness constraint, (ii) a feasibility constraint, (iii) an execution time constraint, or (iv) any combination thereof, and the query is in a first programming language; identifying, from a set of intents, an intent of the query based on one or more key terms within the query; performing a dry run of the query on at least a subset of the plurality of data stores to evaluate, for each data store of at least the subset, whether the data store satisfies the one or more constraints based on the intent and data store metadata, wherein the data store metadata comprises data store information associated with each data store of at least the subset; selecting, based on the dry run, an optimal data store from at least the subset of the plurality of data stores; generating a query result for the query, wherein generating the query result comprises: determining whether the query in the first programming language can be executed on the optimal data store, in response to determining the query in the first programming language can be executed on the optimal data store, executing the query on the optimal data store to obtain the query result, and in response to determining the query in the first programming language cannot be executed on the optimal data store: converting the query to a second query in a second programming language that can be executed on the optimal data store, and executing the second query on the optimal data store to obtain the query result; and providing the query result.

The techniques described herein provide improvements in query routing by ensuring constraints including but not limited to freshness, feasibility, divergence and execution time are considered when routing a query. The use of a dry run evaluation that evaluates user and/or entity provided constraints (e.g., freshness, feasibility, divergence) directly addresses limitations in query routing that are often limited to cost-based query routing. Furthermore, by including freshness as a constraint, the query routing techniques described herein ensure that retrieved data meets a user's freshness requirements, which can improve the relevance and timeliness of query results. The use of dynamic query translation additionally addresses challenges in providing a unified method of querying and ensure queries can be executed on any data store within a heterogeneous system that can support the intended functionality of the query. This can not only improve query routing in a heterogeneous system but also reduce user burden by enabling users to query with any programming language query (e.g., SQL, Vector DSL, etc.) they desire. Additionally, the implementation of a CDL can allow users to explicitly define constraints and priorities for their queries. This can enable query routing to adhere to the priorities of users when routing queries rather than routing solely based on the requirements and/or status of the system.

As used herein, when an action is “based on” something, this means the action is based at least in part on at least a part of the something.

As used herein, the terms “similarly”, “substantially,” “approximately” and “about” are defined as being largely but not necessarily wholly what is specified (and include wholly what is specified) as understood by one of ordinary skill in the art. In any disclosed embodiment, the term “similarly”, “substantially,” “approximately,” or “about” may be substituted with “within [a percentage] of” what is specified, where the percentage includes 0.1, 1, 5, and 10 percent.

### Overview of Clinical Digital Assistant Architecture

Various types of entities (in this context an entity refers to a person, computing device or system, or software, e.g., users, applications, services such as SaaS, digital assistant systems, database subsystems, etc.) may access a data storage system as described above. In many instances, a heterogeneous data system with disparate data stores that provide different combinations of functionality and data access can be useful to improve application and service workflows and to provide end users with a better experience. A particular example of an environment that can interact with a data storage system to improve functionality and end user experience is in health care environments for accessing clinical data.

Providing healthcare to patients typically requires a healthcare provider (e.g., a physician, nurse professionals, other healthcare professionals, etc.) to repeat a number of common tasks for each patient. For example, regardless of the specific reason for an interaction between a healthcare provider and a patient, or the condition of a given patient, the healthcare provider must typically document the patient interaction. For example, the healthcare provider may record the patient interaction in a subjective, objective, assessment, and plan (SOAP) note, or may enter information gained during the patient interaction into a patient record. The healthcare provider may also engage in various other tasks directly or indirectly related to administering healthcare to the patient, such as requesting additional patient information in the form of charts or images, calling in patient prescriptions, and calendaring future tasks, events, and associated reminders.

Performing such healthcare tasks according to known and commonly used methods can be time consuming. In fact, given the typically high volumes of patient encounters, healthcare providers often spend a considerable portion of their workday documenting patient interactions and associated medical information, which reduces the amount of time available to the healthcare provider to administer actual patient care or perform other more critical tasks. For example, healthcare providers may spend considerable time on a daily basis typing or manually entering patient information into electronic health record (EHR) systems. In addition to being time consuming, this process can be tedious, and prone to errors such as but not limited to typographical errors, which can result in inaccuracies and inconsistencies in patient records, and can potentially compromise patient safety and the quality of care provided. Traditional EHR systems can also have complex interfaces any may be difficult to navigate, which can increase the time required for healthcare providers to complete such repetitive tasks and generally frustrate the process Traditional EHR system devices may also be cumbersome to operate, and a lack of intercommunication between such devices prevents a healthcare provider from switching between devices while in the process of performing a task even if doing so would be more efficient. These issues may negatively affect patients as well as healthcare providers. For example, patient information may often be retrieved for review or discussion during a patient interaction or recorded during a patient interaction to ensure accuracy. When the process for retrieving or recording such information is inefficient, as is often the case when performed using traditional systems and methods, it can disrupt the natural flow of the patient interaction and may result in a less seamless and less fulfilling experience for the patient. The tedium and time requirements associated with repetitively performing these tasks can also contribute to healthcare provider burnout. Furthermore, such tasks require consistent and accurate access to data, and steps taken to ameliorate and improve task performance (e.g., through automation or otherwise) must also guarantee accurate and consistent access to clinical and/or patient data.

A digital assistant can be implemented using a clinical digital assistant (CDA) framework as described below to improve workflows and capabilities for healthcare providers. The CDA framework interacts with end users and backend systems to enhance healthcare workflows by integrating APIs, multi-modal user interface (UI), and Electronic Health Record (EHR) data sources. End users (e.g., healthcare providers) may interact with the CDA through natural language-based conversational experiences. The CDA framework includes generative model (e.g., LLM) based agents that can perform specific functionality (e.g., as defined by a plugin, service level logic, etc.) to provide specialized AI capabilities. In response to a user input, an agent can perform one or more actions including, but not limited, to UI actions that enable conversational interaction against a UI element (e.g., filtering content, adjusting visualization), API actions, and data actions (e.g., retrieving relevant data from a data system). To provide access to internal and external knowledge sources including longitudinal records of a patient and domain-specific knowledge, the CDA framework can include a healthcare semantic index. The healthcare semantic index is a heterogeneous data storage system described above that stores and indexes data, such as patient data, and can enable generative model-based agents to reason across knowledge and data sources through natural language metadata (e.g., as stored in semantic objects) and clinical embeddings (e.g., numeric representations) of unstructured text, images, and discrete data. Access to such data can be important in healthcare settings. For example, a physician performing a chart review may need knowledge about relevant drugs for a condition, interactions between drugs, and interactions between drugs and foods in addition to patient-specific data, such as treatment history.

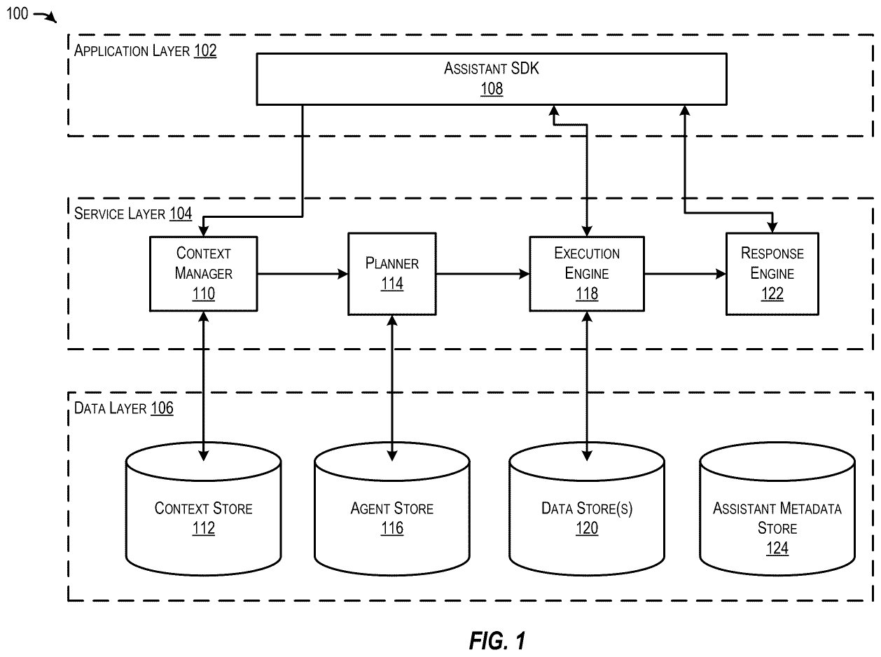

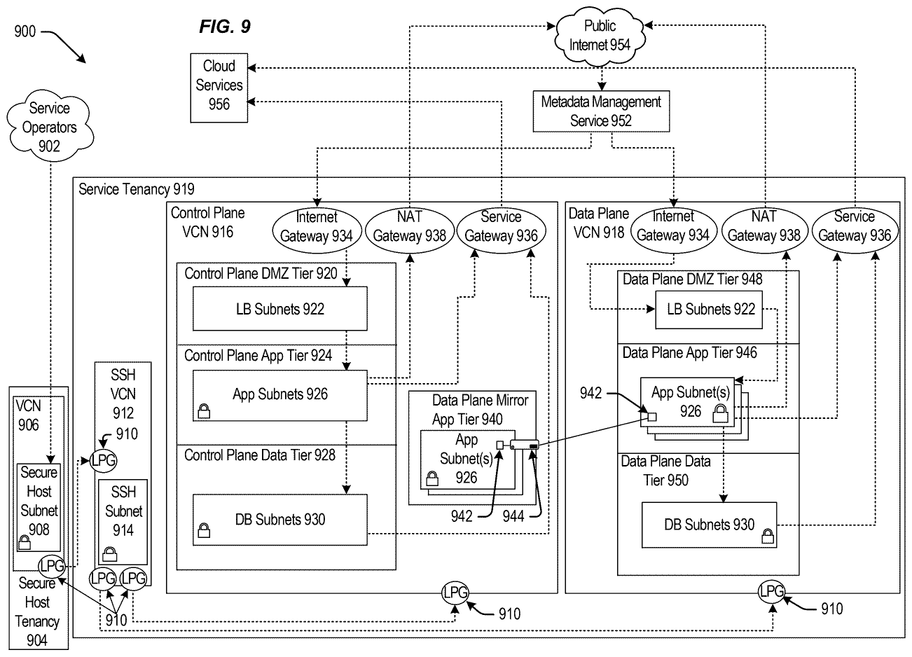

FIG. 1 is an example of an architecture for a computing environment 100 for a clinical digital assistant in accordance with various embodiments. The computing environment 100 can include additional components, fewer components, or different components. In some instances, the computing environment 100 is part of an Infrastructure as a Service (IaaS) cloud service (described in more detail with respect to FIGS. 9-13) and the clinical digital assistant can be implemented as part of the IaaS by leveraging the scalable computing resources and storage capabilities provided by the IaaS provider to process and manage large volumes of data and complex computations.

The computing environment can include various layers including an application layer 102, service layer 104, and data layer 106. Each layer may include components that interact to provide a healthcare workflow as described above. The data layer 106 can be or can include a healthcare semantic index. The application layer 102 can include an assistant software development kit (SDK) 108 that can process user inputs provided by a user through an interface (e.g., a user interface, voice interface, etc.) of an application shell. Examples of user inputs include, but are not limited to, user speech commands, user text commands, user clicks, etc. Additionally or alternatively, the assistant SDK 108 can receive inputs via backend events generated in response to user interactions (e.g., user click events, backend changes, etc.). The assistant SDK 108 can be configured to interact with various components of the service layer 104 (e.g., providing user inputs to the service layer 104, receiving responses from the service layer 104, etc.).

The application layer 102 provides user inputs to a context manager 110 of the service layer 104. The context manager 110 prepares contextual information that can be utilized by components of the service layer 104 to generate a relevant response to the user input and/or user action. The context manager 110 retrieves one or more contexts from a context store 112. A context can act as a holder object for metadata associated with contextual information related to a conversation history, session history, previous executions, etc. The context store 112 can store contexts including, but not limited to, user context, application context, session context, etc. Additionally or alternatively, the context manager 110 may retrieve metadata from an assistant metadata store 124. The assistant metadata store 124 may store metadata for semantic objects and/or plugins that define one or more agents and can be used to identify and select agents and/or actions based on the user input.

The context manager 110 provides contextual information to a planner 114. The planner 114 can be or can utilize one or more generative models (e.g., LLMs or LMMs) fine-tuned to create an execution plan with specified parameters either from a user input (e.g., an utterance), the action performed by the user, the context, or any combination thereof. The execution plan identifies one or more agents and/or one or more actions for the one or more agents to execute in response to the and/or action performed by the user.

The planner 114 can include a retrieval component that retrieves candidate agents and/or actions from the agent store 116. The retrieval component may execute a query on indices of an agent store 116 based on the user input and/or action performed by the user. In some instances, the retrieval component performs a semantic search using words from the user input and/or representative of the action performed by the user. The semantic search uses NLP and optionally machine learning techniques to understand the meaning of the user input and/or action performed by the user and retrieve relevant information from the data layer 106. In contrast to traditional keyword-based searches, which rely on exact matches between the words in the query and the data in the data layer 106, a semantic search takes into account the relationships between words, the context of the query and/or action, synonyms, and other linguistic nuances. This allows the clinical digital assistant to provide more accurate and contextually relevant results, making it more effective in understanding the user's intent in the utterance and/or action performed by the user. The planner 114 can use the candidate agents and/or candidate actions retrieved from the agent store 116 and context determined by the context manager 110 to generate an execution plan listing and/or describing actions that can be executed based on the user input. For example, the planner 114 can determine parameters for the selected action(s) and include the parameters in the execution plan.

The execution plan is transmitted to an execution engine 118 configured to execute the actions of the execution plan. For example, for API actions, the execution engine 118 may execute one or more API calls. For UI actions, the execution engine 118 may populate properties needed to execute the action. For data actions such as knowledge retrieval, the execution engine 118 can execute a query against the data layer 106 (e.g., on one or more data store(s) 120) to retrieve data relevant to the user input. In some examples, to execute a data action, the execution plan can include a semantic search as described that can be executed by the execution engine 118 on the data store(s) to identify relevant information or data (e.g., clinical data related to a certain concept, etc.).

The data layer 106 can be a heterogeneous and disparate data environment as described above. The data layer 106 can include one or more data store(s) 120 that can store patient data (e.g., patient notes, patient discrete data, etc.) and patient agnostic data (e.g., drug information, disease information, drug interaction databases, etc.). The data store(s) 120 can store structured and unstructured data based on the combination of data store(s). For example, the data store(s) 120 can include a relational database, a vector database, an object store, or combinations thereof. One or more of the data store(s) 120 may store clinical embeddings (e.g., in a vector database) to embed knowledge that can be accessed by the service layer 104 and raw data can be enriched by linking information to code-sets (e.g., SNOMED, ICD-10, etc.). Clinical concepts can be stored as semantic objects within the data store(s) 120. The data layer 106 can include data that is kept consistent with a source EHR system. For example, patient data stored in the data layer 106 may be kept consistent with a one or more databases of traditional EHR system through a data ingestion process. As such, changes to patient data made on an external system (e.g., a traditional application used by healthcare providers) can be propagated to the data layer 106 to ensure patient data is accurate irrespective of where the changes are made.

Execution output(s) generated by the execution engine 118 (e.g., data retrieved from the data store(s) 120, API responses, etc.) is transmitted to a response engine 122. The response engine 122 can be or can utilize one or more generative models (e.g., LLMs or LMMs) to generate a response to a user. The response can be a multi-modal response that combines response from different executions into a final response. For example, the response can be text, images, tables, UI elements, action executable by the assistant SDK 108, etc. Response(s) generated by the response engine 122 are transmitted to the assistant SDK 108. The assistant SDK 108 can transmit the response(s) to an application shell to provide the response to the user (e.g., via a user interface, voice interface, etc.).

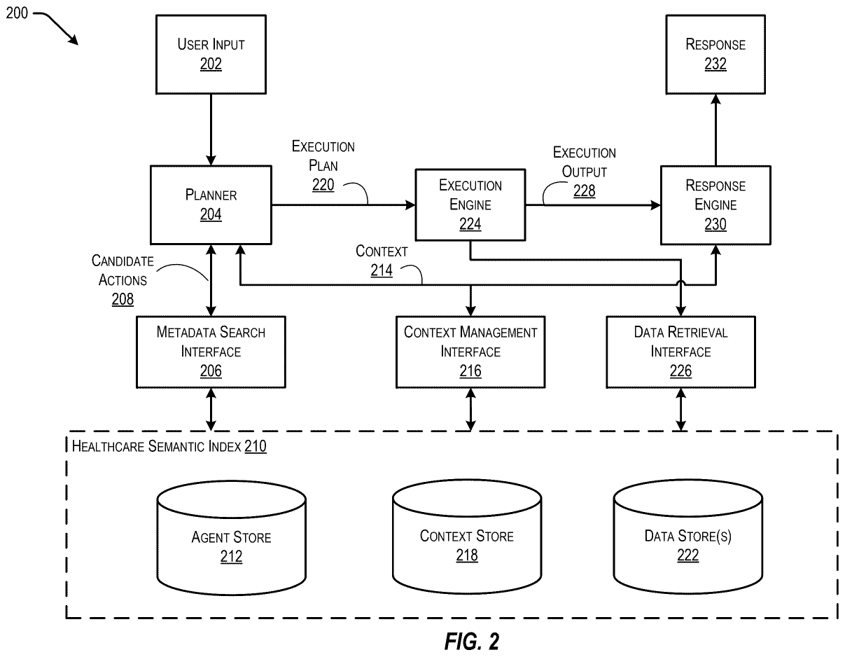

FIG. 2 is a block diagram of a digital assistant runtime flow 200 with components and interfaces into a semantic index, in accordance with various embodiments. As illustrated in FIG>2, A user input 202 can be provided to a planner 204 (e.g., planner 114 of FIG. 1). The user input 202 can be a natural language utterance, user interface action, programming language query, or other forms of user inputs. In this walkthrough, it is assumed that the user is a healthcare provider interested in knowing medical data of a patient. The healthcare provider provides the following input: Has the patient's total cholesterol level ever been over 180?

Based in the user input 202, the planner 204 accesses a metadata search interface 206 to retrieve appropriate candidate actions 208 from the healthcare semantic index 210 (e.g., data layer 106 of FIG. 1). The candidate actions 208 may be retrieved from an agent store 212 that stores a set of actions associated with one or more agents. Candidate actions 208 can be potential actions determined to meet a confidence threshold for a potential topic related to the user input 202. In some examples, candidate actions 208 can be determined by executing a semantic search on the agent store 212 and identifying actions that satisfy a similarity threshold. Examples of candidate actions 208 include, but are not limited to, UI actions, API actions, data actions, etc. For the above input provided by the healthcare provider, the candidate actions can include actions such as getObservations, getVitals, displayChart, etc. which may each be predefined actions associated with UI changes, data retrieval, API execution, etc.

The planner 204 retrieves context 214 containing contextual information related to the conversation history via a context management interface 216. The context 214 is retrieved from a context store 218 and can include contextual information based on a conversation history and/or session history between the healthcare provider and digital assistant. For example, the context 214 can include a patient id, a current time, previous user utterances, previous responses, etc. For the example of the user input provided by a healthcare provider above, the context 214 can identify the patient referenced in the healthcare provider's input as having a patient identifier value of ‘123’ based on information associated with the session and/or previous interactions (e.g., utterances) between the healthcare provider and the digital assistant.

Based on the retrieved candidate actions 208 and context 214, the planner 204 generates an execution plan 220 that can be executed to answer the healthcare provider's question. The planner 204 selects the most appropriate candidate action of the retrieved candidate actions 208. For the above example, the planner 204 may select the getObservations action to retrieve the patient observations from the healthcare semantic index 210. Additionally, the planner 204 may determine parameters needed to execute the selected action. The parameters can include, for example, an API payload or a query that can be executed on one or more data store(s) 222 of the healthcare semantic index 210. As described with respect to FIG. 1, the planner 204 can be or can make use of one or more LLMs to generate the execution plan 220. In some examples, the candidate actions 208 and context 214 may be provided as a prompt to the planner 204 and/or one or more generative models used by the planner 204 to generate the execution plan 220. For the above example user input 202, the parameters can include a query that can be executed on the healthcare semantic index 210 to retrieve the patient's cholesterol level. A generated execution plan 220 can be as follows:
Action: getObservations
Parameters:
query: SELECT*FROM Observations WHERE vitalSigns=‘Total Cholesterol’ AND patientID=‘123’ and value >180

The planner 204 provides the execution plan 220 to an execution engine 224 (e.g., execution engine 118 of FIG. 1). The execution engine 224 executes the execution plan 220 to generate an execution output 228. For data actions, the execution engine 224 can execute the execution plan 220 via a data retrieval interface 226. The data retrieval interface 226 can be a programmatic interface for query execution on the healthcare semantic index 210. In some implementations, the data retrieval interface 226 can be or can include one or more API endpoints. Data for a query in the execution plan 220 can be retrieved from one or more data store(s) 222 of the healthcare semantic index 210. The data store(s) 222 can include clinical data stores and may include data stores of various types, including but not limited to relational databases, vector databases, etc.

An execution output 228 generated by the execution engine 224 based on the execution of the execution plan 220 is provided to a response engine 230. The execution output 228 can include data retrieved by execution the execution plan 220, an output of an API call, an action to be performed by an application (e.g., a UI action), references to sources of data and/or outputs, or combinations thereof. The response engine 230 can generate a rich output with appropriate data elements in the output. The response engine can be or can make use of one or more generative models to generate the response 232 that is provided to the user. The response can be an event that is provided to a user, multi-modal response, references, a query result, etc., generated based on the execution output 228 of the execution engine 224. Additionally or alternatively, the response engine 230 can retrieve context 214 (e.g., as retrieved and used by the planner 204) to generate the response 232 with contextual information. For example, the response engine 230 may determine that the name of the patient with patient id ‘123’ as identified above is “Grace” and may include the patient's name in the response 232. A response to the healthcare provider with the above question can be a text and tabular response as follows:
Grace's total cholesterol level was reported to be over 180 mg dL in the last 2 Lipid Panels.

| Type | Date | Results |
|---|---|---|
| Lipid Panel | Feb. 17, 2024 | Total: 220 mg/dL (elevated) |
| | | HDL: 60 mg/dL (normal) |
| | | LDL: 150 mg/dL (elevated) |
| Lipid Panel | Nov. 17, 2023 | Total: 230 mg/dL (elevated) |
| | | HDL: 60 mg/dL (normal) |
| | | LDL: 160 mg/dL (elevated) |

### Overview of Semantic Index

A Semantic Object Model (SOM) can be an effective way of abstracting data to provide a unified view of data that transcends limitations of individual data storage methods, models, schemas, etc. within a data storage system. A semantic object stored within a data system can represent a particular concept and include various attributes associated with the particular semantic object. In some implementations, the semantic object can include self-describing metadata and/or linked actions for custom operations. The semantic object metadata can be used by a model (e.g., a generative model such as an LLM, etc.) to query the model.

Semantic Index (SI) is a data storage system implementing a Semantic Object Model including disparate and varying types of data stores. SI can store data in a custom way spanning multiple storage systems, abstracting from applications and providing a unified, durable, consistent and powerful data store. As a non-limiting example, SI can be used in healthcare environments (e.g., as described above with respect to the data layer 106 of FIG. 1 and healthcare semantic index of FIG. 2) to improve data accessibility for healthcare professionals and improving patient treatment. Semantic objects within SI can reflect clinical concepts (e.g., patients, treatments, observations, etc.). SI can maintain consistency with a primary source of truth (e.g., an electronic health record (EHR) system of record) to provide patient data related to medical history, diagnoses, etc. Many legacy applications used by healthcare providers rely on prominent platforms supporting conventional EHR systems of record for managing and interfacing with patient and clinical data. For new applications, it can be beneficial to introduce improved semantic techniques to improve access to patient and clinical data. However, for patient records to remain consistent across data platforms and applications, it is important the patient records remain consistent across data models and systems.

In some embodiments, a digital assistant, or chatbot, can interface with the Semantic Index to enable a user to query patient history and data more efficiently. For instance, a digital assistant may be able to query SI using semantic queries generated by a generative model (e.g., a Large Language Model (LLM), etc.). A user may interact with the digital assistant using natural language and then convert the reactions into intelligible queries, such as for clinical questions, etc.

In the interest of clarity of explanation, embodiments of the present disclosure are described in connection with particular data storage systems (e.g., Semantic Index), services (e.g., digital assistants), data models (e.g., Semantic Object Model, relational data models, etc.). However, the embodiments are not limited as such and instead, similarly, and equivalently apply to any data storage system, data models, and services in a multi-data store environment.

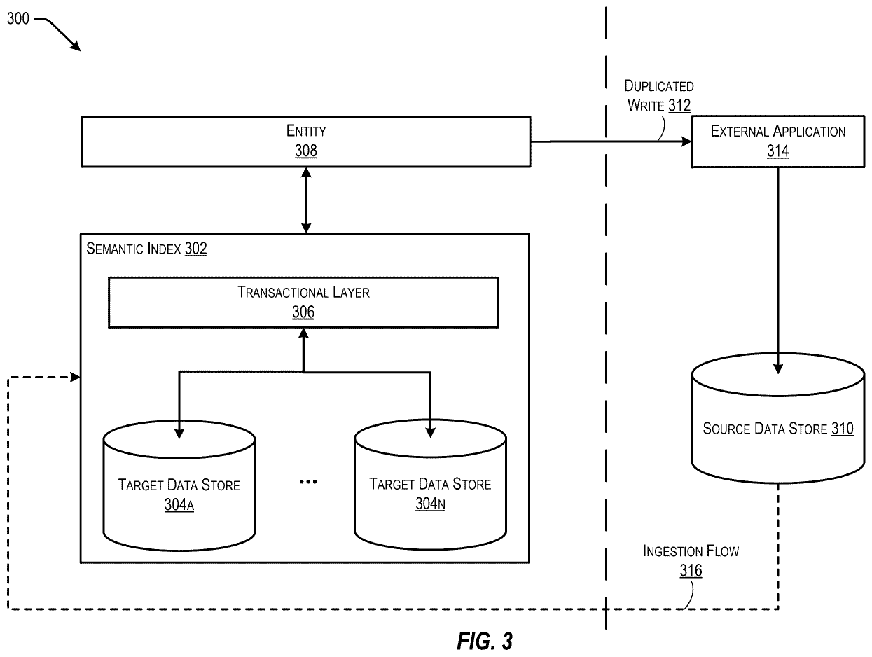

FIG. 3 is a simplified block diagram of an environment 300 of a distributed storage system incorporating Semantic Index. In some instances, the computing environment 300 is part of an Infrastructure as a Service (IaaS) cloud service (as described in more detail with respect to FIGS. 9-13) and semantic index can be implemented as part of the IaaS by leveraging the scalable computing resources and storage capabilities provided by the IaaS provider to process and manage large volumes of data and complex computations. Environment 300 includes Semantic Index (SI) 302 implementing protocols for semantic retrieval of data as described above. While the description of this figure may include various components and processed, it should be understood that additional components, fewer components, or different components as described can be implemented to provide the desired impact.

Semantic Index (SI) 302 can include multiple data stores (e.g., target data store 304a, target data store 304b). In some examples, one or more data stores of target data stores 304a-n are database(s) deployed in a cloud environment using an IaaS cloud service (e.g., as described in more detail with respect to FIGS. 9-13). Each data store within SI 302 may be a different type of data store. For example, target data store 304a can be a vector database (e.g., OpenSearch, Pinecone, etc.) and target data store 304b can be a relational database (e.g., Oracle, MySQL, PostgreSQL, etc.). Additionally or alternatively, Semantic Index 302 can include data stores including, but not limited to, a graph database, NoSQL database, key-value stores, message queues, object stores, etc. Target data stores 304a-n may each contain copies of the same data but provide multiple methods to query and access the data.

While target data stores 304a-n may each be the same and/or different type of data store, each target data store 304a-n may follow the same schema and/or data model. For example, SI 302 can implement the Semantic Object Model as described above, and each target data store 304a-n may implement a schema compatible with the Semantic Object Model. As a particular example, target data store 304b can be a relational database that implements semantic objects as tables within the target data store 304b. Relationships between semantic objects in a relational database may be represented as foreign keys reflecting references to other tables within the target data store 304b.

SI 302 includes a transactional data layer 306 (e.g., data retrieval interface 226 of FIG. 2) that can process queries to SI 302. The transactional layer 306 can support various types of queries, including, but not limited to QDSL, SQL, ingestion from external sources, etc. Additionally or alternatively, the transactional data layer 306 provides a software development kit (SDK) and/or application programming interface (API) that enables an entity 308 (e.g., a user, application, digital assistant, etc.) to interact with Semantic Index 302. For example, the transactional layer 306 includes an API allowing the entity 308 to read and/or write data to SI. The transactional layer 306 can act as an abstraction of the data stored in SI to the entity 308. For example, the entity 308 can call the API to request access to certain data without having knowledge about specific implementations of data models, schemas, and/or data stores within SI 302. Alternatively or additionally, the entity 308 can query SI using a SQL statement, a vector search, or the like. As such, the entity 308 can query and write to SI based on their own internal data models and/or schemas without understanding specifics about the data storage implementations in SI 302. As a particular example, the entity 308 can be a component of a digital assistant system (e.g., planner 204 of FIG. 2) with the capability to receive natural language utterances from a user and determine an execution plan including the execution of one or more programming language queries to retrieve data for addressing and/or responding to the utterances.

In the environment 300 depicted in FIG. 3, writes to SI 302 can occur as a direct write by the entity 308 and/or ingested writes propagated from a source data store 310. The source data store 310 may be a data store externally managed by another organization and/or located in a separate data environment. The source data store 310 may implement a different schema and/or data model than SI 302 and target data stores 304a-n. In some implementations, to maintain consistency between data stored in the target data stores 304a-n and the source data store 310, each direct to SI 302 by the entity 308 may be duplicated to the source data store 310 via a duplicated write 312 provided to an external application 314. The external application 314 may execute the duplicated write 312 on the source data store 310. As an example, in healthcare environments (e.g., as described above with respect to FIGS. 1-2), the source data store 310 can be a database associated with an EHR system. A direct write to SI 302 can include changes to patient data in SI 302. Such changes to patient data are duplicated to the EHR system by providing the duplicated write 312 to the external application 314 (e.g., an application traditionally accessed by a doctor to update patient data) to ensure patient data is consistent.

SI 302 can maintain consistency between the target data stores 304a-n and the source data store 310 via an ingestion flow 316. Data stored in the source data store 310 may be replicated and concurrently stored in the target data stores 304a-n. In some instances, target data stores 304a-n can include data not stored in the source data store 310. For example, SI 302 may store summaries for semantic objects (e.g., stored within a metadata store in SI) that are not compatible with the schema and/or data model implemented by the source data store 310.

Writes to SI can be writes propagated from SI. For example, the external application 314 may execute a direct write on the source database (e.g., a doctor may use PowerChart to update patient data). In eventually consistent models, writes to the source database should be propagated to the target database and, accordingly, such writes can be ingested by SI 302 through the ingestion flow 316. The ingestion flow 316 can be or can include an event stream, change data capture (CDC) system, replication system, or similar that can capture changes in the source database and replicate the changes in a write to SI.

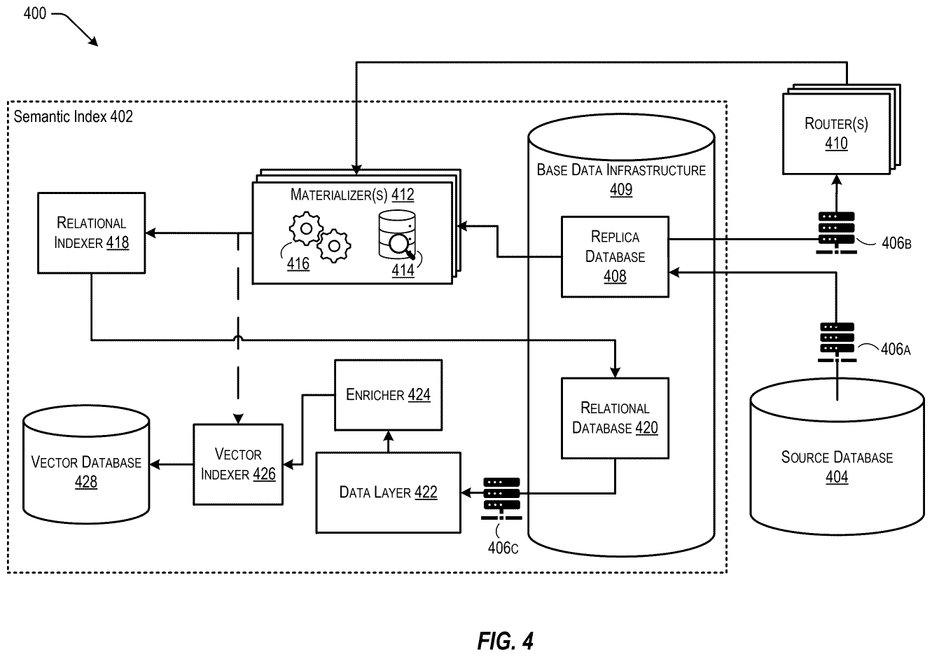

FIG. 4 is an example of an architecture for a computing environment 400 for semantic index implemented with disparate data stores. Certain aspects of FIG. 4 are described with respect to components of the environment described with respect to FIG. 3. As illustrated in FIG. 4, an infrastructure and various services and features can be used to enable the system as described. The following is a detailed walkthrough of an ingestion flow (e.g., ingestion flow 316 of FIG. 3) and the role and responsibility of the components, services, models, and the like of the computing environment 400 within an ingestion flow. In this walkthrough, it is assumed that Semantic Index (SI) 402 is a data storage system that includes data consistent with a source database 404. It is also assumed that any writes to SI 402 are also applied to the source database. In this example, the source database 404 implements a different schema than SI 402 and SI 402 implements a Semantic Object Model.

While the embodiment of computing environment 400 in FIG. 4 illustrates a particular ingestion flow, this is not intended to be limiting and is merely provided to facilitate a better understanding of the role and responsibility of the components, services, models, and the like of the computing environment 400 within the ingestion flow. Some embodiments may include more components than depicted, less components than depicted, or different components than depicted. The ingestion flow, as described, can enable consistent and scalable replication across disparate data stores to enable data synchronization between a source data system and a target data system.

The computing environment 400 includes a source database 404. As described with respect to FIG. 1, the source database 404 can act as a primary source of truth for SI 402. The source database 404 can be a relational database, vector database, NoSQL database, etc. Data stores within semantic index 402 are made consistent with the source database 404. In some implementations, the semantic index 402 includes data not included in the source database 404. As a non-limiting example, the source database 404 is a relational database and acts as an electronic health record (EHR) system of record. The source database 404 may implement a particular schema that is conventionally known.

The source database 404 (e.g., source data store 310 of FIG. 3) can receive a write. For example, a SQL statement may be executed on the source database 404. As described in FIG. 1, the source database can receive the write directly from an external application, or as a duplicated write from a direct write to the Semantic Index 402. By writing the data to the source database, one or more data operations are performed on the source database 404 (e.g., an id is updated, a value is deleted, etc.).

A change data capture (CDC) system 406a (e.g., Kafka, Oracle GoldenGate, Debezium, etc.) may capture data changes in the source database 404 and transmit the data changes to a replica database 408 maintained in semantic index 402. The change data capture system 406a may extract data changes from a transaction log (e.g., redo logs, write-ahead logs, etc.) maintained by the source database 404. The data changes can be transmitted to the replica database 408 as a transaction including one or more data operations (e.g., insertions, deletions, updates, etc.) in the source database 404. In some examples, the data changes can be captured and transmitted as an event stream. The replica database 408 may be a copy of the source database 404 maintained within SI 402 and can serve as the most current known state of the source database 404. The replica database 408 can implement and follow the same schema and/or data model as the source database 404. As such, data changes captured by the CDC system 406a may be executed on the replica database 408 exactly as received. The CDC system 406a can maintain an order of commit of operations executed on the source database 404 and data operations can be executed on the replica database 408 in the order determined by the CDC system 406a.

A second CDC system 406b (e.g., a second Oracle GoldenGate, Debezium, etc.) can capture data changes executed on the replica database 408. The type of CDC system 406b may the same or different as the type of CDC system 406a. The CDC system 406b may extract the data changes from a transaction log maintained by the replica database 408. CDC system 406b packages data changes in the replica database 408 and transmits the data changes to one or more router(s) 410. In some examples, a CDC payload including one or more data operations may be added to a queue associated with the router(s) 410.

The router(s) 410 can be implemented using software only, hardware only, or any combination thereof. The router(s) 410 can be configured to determine semantic objects impacted by data changes in the source database 404 and replica database 408. In some examples, each router 410 may include a mapping of a schema and/or data model implemented by the source database 404 and the schema and/or data model implemented by SI 402. For example, the router(s) 410 may maintain a schema mapping between a table in the source database 404 and semantic objects in SI 402 that consume one or more attributes from the source database 404 table. Accordingly, upon identifying a change to the table in the source database 404, the router(s) 410 may determine the semantic objects impacted by the table update. The router(s) 410 may have a base understanding of attributes and/or fields associated with a particular semantic object. However, each semantic object may include nested structures that the router may be unable to fully and/or accurately map.

The router(s) 410 may not maintain full knowledge of all attributes and nested structures associated with each semantic object in SI 402. For such examples, the router(s) 410 may be configured to identify impacted semantic objects based on table updates, but may not be able to properly construct semantic objects according to the schema implemented by SI 402. The router(s) may invoke one or more materializer(s) 412 to construct the identified impacted semantic objects. The materializer(s) 412 can be implemented with software, hardware, or a combination thereof. Each materializer of the one or more materializer(s) 412 may be configured to construct a particular semantic object. For example, a first materializer may be configured to construct a patient semantic object based on a definition of a patient concept in the semantic model. A second materializer may be configured to construct a treatment semantic object. Upon determining an updated table from the replica database 408 impacts a patient semantic object, the router(s) 410 can invoke the first materializer configured to construct the patient semantic object to generate an updated patient semantic object. The router(s) 410 may invoke multiple materializers by providing each materializer with instructions to construct an updated semantic object. Each materializer of the materializer(s) 412 may construct their respective semantic objects in parallel, sequentially, or any combination thereof.

Each materializer 412 can include a view collector 414 and a finalizer 416. The view collector 414 retrieves current data (e.g., parameters, attributes, data values, etc.) associated with the semantic object based on a known structure of the semantic object. In some examples, the view collector 414 can be a view that presents data from the replica database 408 in a relational and/or JSON format. The finalizer 416 includes software, hardware, or combinations thereof, configured to construct the semantic object using the information retrieved by the view collector 414 from the replica database 408. The semantic object can be subsequently written to the relational database 420. The relational data store can follow the SOM, and each semantic object may be stored in a particular table related to the corresponding semantic object. The semantic object is indexed by a relational data store. In some examples, the finalized semantic object generated by the materializer(s) 412 is provided to a relational indexer 418. The relational indexer 418 may optimize data retrieval and may provide a mechanism for writing the semantic object into its relational shape in the relational database 420. In some examples, the relational indexer 418 may provide pointers to particular rows in the relational database 420 to optimize writes to the relational database 420. Accordingly, semantic objects constructed by the materializer(s) 412 can be written to the relational database 420. The replica database 408 and relational database 420 may be hosted on a base data infrastructure 409. The base data infrastructure 409 may represent the primary source of truth within SI 402, and data stores hosted outside the base data infrastructure 409 may maintain consistency with the base data infrastructure 409.

A third change data capture (CDC) system 406c can capture data changes in the relational database 420. For example, data transactions executed on the relational database 420 to write a finalized semantic object can be reflected in a transaction log associated with the relational database 420. Data changes in the relational database 420 may be associated with an updated semantic object. The CDC system 406c may extract change data from the transaction log associated with relational database 420. The change data can be transmitted to a data layer 422. The data layer 422 may mirror a read-write interface provided by an SDK associated with SI 402 and may orchestrate read-write requests to involve processes such as authorization, persistence, versioning, and event management. In some examples, the data layer 422 may execute processes such as versioning and event management asynchronously.

The change data (e.g., updated semantic object(s) and/or updates associated with one or more semantic object(s)) can be processed by an enricher 424 configured to add context to the data and prepare the data to be stored in the vector database 428. Semantic object data determined from the change data can be vectorized and provided to a vector indexer 426. The vector indexer 426 can provide a mechanism for writing a semantic object in its vectorized shape into the vector database 428. In some examples, the semantic object may be stored as one or more embeddings to capture semantic meaning and relationships across semantic objects.

While the ingestion flow depicted in FIG. 4 depicts a data write executing on the relational database 420 store prior to being converted and indexed to be written to the vector database 428, the finalized semantic object generated by the materializer(s) 412 directly to the vector indexer 426 to be written to the vector database 428. In such ingestion flows, each semantic object may be written in parallel to the relational database 420 and the vector database 428.

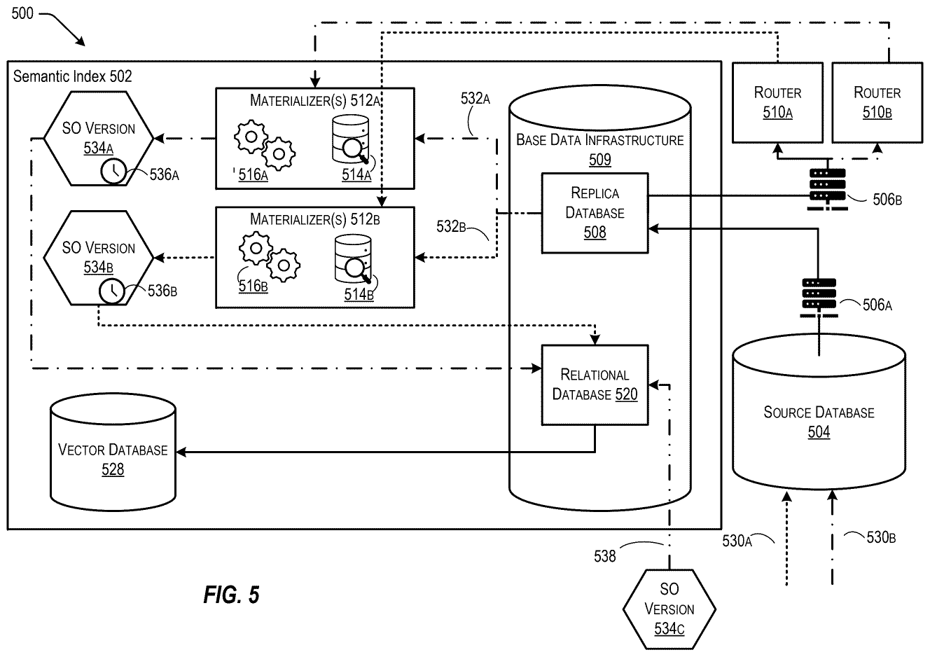

FIG. 5 depicts an ingestion flow 500 implementing watermark generation for replicating multiple data writes from a source system to a target system, in accordance with various embodiments. Certain aspects of FIG. 5 are described with respect to components of the computing environments described with respect to FIG. 4. While the ingestion flow 500 describes watermark generation with respect to writes to the relational database 520 within SI 502, the ingestion flow 500 can include watermark generation for additional target stores within SI 502 such as vector database 528 or any additional target data store not depicted in FIG. 5. Each target data store of SI 502 may maintain distinct sets of watermarks. Additionally or alternatively, source database 504 or other similar source systems may include similar and/or different implementations of watermarking.

At the semantic object level, watermarks can be stored as attributes and/or metadata of a sematic object (e.g., as a timestamp, etc.). A watermark can indicate the freshness of the semantic object and a time up to which the data can be considered accurate. Watermark generation may vary depending on the source of the data write within the data system.

For data writes ingested by SI 502 from a source data system (e.g., source database 504), watermarks can be determined by a materializer configured to generate a particular semantic object. As an example, a source data write 530a is executed on the source database 504. As described with respect to FIG. 4, the source data write 530a is extracted by a CDC system 506a (e.g., CDC system 406a of FIG. 4) and executed on the replica database 508 (e.g., replica database 408 of FIG. 4). The source data write 530a is then processed by a CDC system 506b (e.g., CDC system 406b of FIG. 4) and provided to a router 510a (e.g., router(s) 410 of FIG. 4), which routes the transaction generated by CDC system 506b to a relevant materializer 512a (e.g., materializer(s) 412 of FIG. 4) that can generate an SO version 534a. As used herein, a version of a semantic object can be an instance, data record, etc. of a semantic object that can be stored in a data store (e.g., as generated by a materializer).

The materializer 512a generates the SO version 534a by executing one or more data read(s) 532a on the replica database 508 to retrieve current state information of attributes of the identified semantic object. The materializer 512a can determine a watermark 536a associated with the SO version 534a based on a time at which the one or more data reads 532a were executed. In some examples, the one or more data reads 532a are initiated and/or executed by a view collector 514a (e.g., view collector 414 of FIG. 4) configured to retrieve a current state of data relevant to the semantic object. In some examples (e.g., when the replica database 508 implements a relational data model and/or schema), the view collector 514a may be initialized with one or more predefined Structure Query Language (SQL) queries for creating a view (e.g., virtual table) of the relevant data consumed by the semantic object. The one or more predefined SQL queries for generating a view including data relevant to a particular semantic object can be executed by the view collector 514a, causing one or more data reads 532a to be performed. The watermark 536a can be or can include a timestamp corresponding to the time at which the data read(s) 532a were executed. The timestamp may be based on a time determined by a time determining mechanism of the replica database 508 and/or SI 502. For example, the timestamp may be determined by using a wall clock, logical clock, physical clock, etc. As such, the watermark 536a can reflect the freshness of the data up to the point at which it was read by the materializer 512a.

In some instances, the source database 504 may receive a second source data write 530b that can impact the same semantic object as the source data write 530a. The second source data write 530b may impact the same data within the source database 504 as the first source data write 530a and/or different data within the source database 504. For example, an impacted SO may consume information from a group of tables within the source database 504. The first source data write 530a may update one or more values in a first table within the group of tables. The second source data write 530b may update one or more values in the first table or in a second table within the group of tables. Because the impacted semantic object consumes one or more values from both the first table and the second table, the materializer(s) generate versions of the same semantic object upon receiving the data changes.

Once written to the replica database 508, the first source data write 530a and second source data write 530b may be processed in parallel according to the ingestion flow. For example, a transaction associated with the first source data write 530a may be provided to router 510a by the CDC system 506b and a transaction associated with the second source data write 530b may be provided to a second router 510b. The routers 510a-b may process the respective data writes in sequence, in parallel, or combinations thereof. In some instances, router 510b may finish processing and routing a transaction corresponding to the second source data write 530b before router 510a finishes processing and routing a transaction corresponding to the first source data write 530a despite the first source data write 530a executing on the source database 504 before the second source data write 530b.

Additionally or alternatively, SI 502 may include multiple materializers 512a-512b configured to generate the same semantic object. In some examples, the semantic object generated by the materializers 512a-b may be a semantic object that is identified as receiving frequent updates in the source database 504. The materializers 512a-b may process CDC payloads corresponding to the source data writes in parallel, in sequence, or combinations thereof. Furthermore, the materializer 512a-b may finish processing the payloads in the same order and/or a different order as the processing performed by the routers 510a-b. For example, router 510a may transmit CDC payload(s) to materializer 512a before router 510b transmits CDC payload(s) to materializer 512b, but materializer 512b may generate SO version 534b before materializer 512a generates SO version 534a.

Watermarks determined by the materializers can resolve staleness issues that can be cause by older data writes overwriting new data writes. Each materializer 512a-b generates the respective SO versions 534a-b using information retrieved from replica database 508 from data reads 532a-b. The information retrieved from the replica database 508 can include relevant values for each attribute of the semantic object regardless of whether the value was updated by any particular source data write. As such, the SO versions 534a-b include information about the respective SO from the replica database 508 that is accurate up to the time of the read and can include changes from writes committed after the respective source data writes 530a-b. For example, a third source data write may be executed on the replica database 508 while transactions corresponding to source data writes 530a-b are processed by routers 510a-b and/or before materializers 512a-b retrieve relevant information from the replica database 508 via data reads 532a-b.

The watermarks 536a-b can accordingly reflect the freshness of the semantic object according to the replica database 508 regardless of commit order in the source database 504. For example, the watermark 536b for SO version 534b indicates the SO version is accurate with respect to the source database 504 as of the timestamp reflected by the watermark 536b. The watermark 536a for SO version 534a indicates the SO version is accurate with respect to the source database 504 as of a timestamp reflected by the watermark 536a. SO versions 534a-b can be written to relational database 520 based on a comparison of the watermarks 536a-b and the watermark of the SO as stored in the relational database 520.

Additionally or alternatively, SI 502 can receive a direct data write 538 including changes to a semantic object within a database. The direct data write 538 can be associated with an SO version 534c that may include the same and/or different data as SO versions 534a-b and can result in a stale overwrite depending on commit orders to the source database 504 and to SI 502. SI 502 can generate a placeholder watermark for the SO version 534c corresponding to the direct data write 538. The placeholder watermark may be calculated as a current timestamp incremented by a single unit of time. For example, if the smallest unit of time tracked by SI 502 is a nanosecond, the watermark for SO version 534c may be calculated as the current time incremented by a nanosecond. The SO version 534c can then be written to the relational database 520 according to watermark evaluation of the semantic object version currently stored in the relational database 520. For example, SI 502 can perform a comparison between the watermark of the semantic object as stored in the relational database 520 and the placeholder watermark determined for SO version 534c to determine whether the SO version 534c is associated with fresh data that can safely be written to the relational database 520.

Because SI 502 maintains consistency with the source database 504, one or more duplicated source data write including information associated with SO version 534c can be executed on the source database 504. For example, upon receiving direct data write 538, a duplicated source data write can be generated and executed on the source database 504. The duplicated source data write can subsequently be ingested and generated as described with respect to SO versions 534a-b.

### Constraint-Based Query Routing

As described above, constraint-driven query routing can be important in heterogeneous data storage systems to improve access to data based on constraints associated with a query. Conventional data systems, however, often do not consider constraints such as freshness and feasibility when routing queries and may be limited in heterogeneous data environments with various combinations of disparate data stores. To address these limitations, constraint-driven routing can be implemented to route queries to data stores within a heterogeneous system that can optimally generate query responses based on the constraints associated with a query and a determined intent of the query.

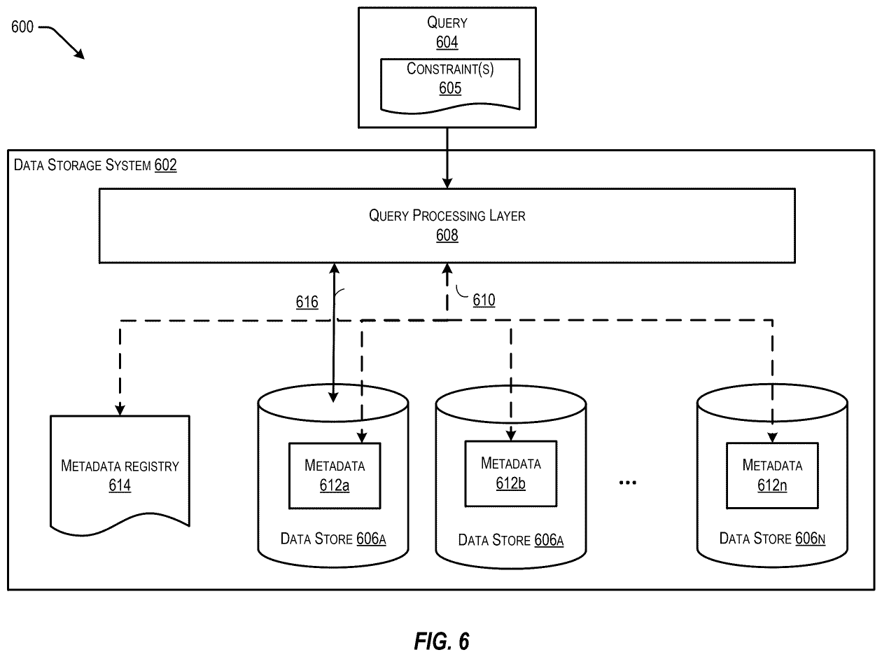

FIG. 6 is a block diagram of a data storage system implementing a constraint-driven query routing with heterogeneous data stores, in accordance with various embodiments. A data storage system 602 (e.g., SI as described with respect to FIGS. 1-5) can receive a query 604 for data stored within the data storage system 602. The data storage system 602 can be a heterogeneous data environment as described above including data stores 606a-n (e.g., target data stores 304a-n of FIG. 3, relational database 420 and vector database 428 of FIG. 4, etc.) of varying types and data models. As examples, data stores 606a-n can include relational database(s), vector database(s), graph database(s), object store(s), etc. The query 802 can be a programming language query (e.g., a Structured Query Language (SQL) query, Query Domain Specific Language (QDSL), API query, etc.). Additionally or alternatively, the query 604 can be a natural language utterance that is translated to a programming language query by one or more components of the data storage system and/or an application accessing data in the data storage system 602. For example, the query 604 can be a SQL query generated based on a natural language utterance provided by a user (e.g., as described with respect to FIGS. 1-2). The query 604 can be or can include a request for one or more data records (e.g., semantic objects) stored in data stores 606a-n.

The query 604 can specify one or more constraints 605 expected to be met by query execution. A constraint can be a condition expected to be met and/or achieved by the query execution. Examples of constraints include, but are not limited to, freshness, feasibility, divergence, and execution time. The constraints 605 associated with the query 604 can be any combination of a freshness constraint, feasibility constraint, execution time constraint, or other constraint supported by the data storage system 602.

A freshness constraint included in the one or more constraints 605 can indicate how fresh data is expected to be. In some instances, the freshness constraint can include evaluations of lag, divergences, or a combination thereof. For example, the freshness of data can be determine based on a last update and correctness of the data. The freshness constraint can be expressed as an age (e.g., in milliseconds, seconds, etc.) of data within a data store. The age can be determined based on when data was updated within the data store. Additionally or alternatively, the freshness constraint can be expressed as staleness threshold. A feasibility constraint indicates whether a data store supports query operations of the query (e.g., as a binary value, etc.). Some data stores may be unable to execute certain query operations. An execution time constraint can indicate a max query latency (e.g., a response time for executing the query and retrieving data). As an example, execution time can be expressed as an amount of time (e.g., milliseconds, seconds, etc.) that would be acceptable for query execution. In some examples, execution time can be predicted based on factors including but not limited to data size, indexes, and system load.

The query 604 may be received at a transactional layer (e.g., data retrieval interface 226 of FIG. 2, transactional layer 306 of FIG. 3) not depicted in FIG. 6. The transaction layer may support read and/or write operations from external sources. As an example, the transactional layer can include one or more API endpoints for an entity (e.g., components of a clinical digital assistant system as described with respect to FIGS. 1-2) to send an API request including the query 604. In such examples, query 604 and/or constraints 605 may be provided as fields within an API payload. For example, the query 604 may be provided in a first field and the constraints 605 may be provided in a second field of a payload.

In some instances, the constraint 605 can be specified using a Constraint Definition Language (CDL) expression. CDL defines a structured set of constraints that can be applied in conjunction or disjunction of individual constraint(s) 605. The CDL can enable entities (e.g., users, CDA systems as described above, etc.) to express query level constraints within the query 604 and/or as a secondary field or parameter provided with the query 604. For example, the CDL expression can be included in a JSON body of a REST API request sent to a transaction layer of the data storage system 602.

The CDL can define logical precedence of the various constraints. Logical precedence can include operators including but not limited to AND, OR, and PRIORITY. For example, an AND operator in the CDL indicates that all constraints specified must be satisfied. An OR operation indicates any of the specified constraints may be satisfied. In some examples, the CDL may specify logical groupings of constraints that are each associated with a different logical precedence. For example, one logical group of constraints within a CDL identified query may be associated with an AND operator and another group may be associated with an OR operator.

A PRIORITY operator can indicate the importance of a certain constraint when making decisions on which data store to route to. For example, constraints with a high priority may be more important in selecting the optimal data store than constraints with a low priority. An example CDL syntax can be as follows:

```
{
  “constraints”: {
    “freshness”: {
      “max_age_seconds”: 300
      “priority”: “high”
    },
    “execution_time”: {
      “max_milliseconds”: 1000
      “priority”: “low”
    },
    “combine_mode”: “AND”
  }
}
```

In some examples, priority values operators for constraints may be numeric values. Additionally or alternatively, priority operates may be mapped to numeric values. For example, a high priority may be assigned a value of 1, a low priority may be assigned a value of 0.5, etc.

The query 604 and associated constraints 605 are provided to a query processing layer 608 that processes the query 604 to select an optimal data store of data stores 606a-n for query execution. The query processing layer 608 can be or can include hardware components, software components, or combinations thereof configured to perform query processing operations including, but not limited to, parsing, optimizing, and executing the query 604.

The query processing layer 608 parses the query 604 and identifies an intent of the query 604. The intent of the query 604 can correspond to a query type and query operations associated with the query type. For example, the intent of the query can be determined from a set of intents including, but not limited to, point queries, filter queries, join queries, aggregate queries, and subqueries. In some examples, key terms of the query 604 (e.g., within a query string) may be used to determine an intent of the query. Key terms can include keywords, clauses, expressions, words, operators, functions, etc. that can be used to uniquely identify an intent. In some examples, the query processing layer 608 performs pattern matching to identify certain key terms within the query. Additionally or alternatively, the query processing layer 608 determines a structure of the query 604 using the key terms (e.g., tokens) and grammatical rules of the query language to identify the intent of the query. The query structure can be used to determine a semantic intent of the query (e.g., an explanation of a meaning and/or purpose of a query). Table 1 lists example intents and key terms that may be used to identify the intents (e.g., by performing pattern matching, identifying query structure, etc.). The examples are not intended to be limiting and can include additional, fewer, and/or different intents and/or key terms.

TABLE 1

| Intent | Intent Meaning | Example Key Terms (SQL) |
|---|---|---|
| Point | Direct lookup based on | SELECT clause with |
| Query | primary key or indexed | primary key |
| | attribute | |
| Filter | Condition-based | WHERE clause and no |
| Query | retrieval of records | primary key |
| Join | Combine data across | JOIN clause |
| Query | multiple tables | |
| | or documents | |
| Aggregation | Calculate values (e.g., | Functions: COUNT, SUM, |
| Query | count group, etc.) | AVG, MIN, MAX |
| | over a dataset | Clauses: GROUP BY, |
| | | HAVING |
| Subqueries | Nested queries for | SELECT clause nested |
| | intermediate | in FROM, WHERE, |
| | processing | HAVING clauses |

In some examples, the query processing layer 608 can utilize a machine learning model trained on a set of queries and corresponding intents to predict the intent. Additionally or alternatively, the query processing layer 608 may utilize natural language processing to derive an explanation of the query. For example, a generative model (e.g., an SQL to NL model) may be used to explain a semantic intent of the query.

The query processing layer 608 performs a dry run 610 to evaluate which data store best satisfies the one or more constraints 605 and the identified intent. The dry run 610 is an evaluation of a data store used to determine constraint satisfaction prior to query execution. The query processing layer 608 may perform the dry run 610 on all data stores 606a-n or a subset of the data stores 606a-n. The query processing layer 608 can incorporate system constraints (e.g., resource availability, data store health, etc.) of SI 602 before and/or while performing the dry run evaluation. For example, the query processing layer 608 may determine that data store 606c is not operational based on known resource availability within data storage system 602. Accordingly, the query processing layer 608 may exclude data store 606c from the dry run 610 and perform the dry run on the remaining subset of data stores (e.g., data stores 606a-b and 606d-n). Additionally or alternatively, the query processing layer 608 may determine based on historical query executions that a particular data store of data stores 606a-n does not meet the constraint(s) 605 as specified in the query and may exclude the data store when performing the dry run 610.

The dry run 610 includes evaluating metadata 612a-612n associated with each respective data store 606a-606n. The metadata 612a-612n for each respective data store 606a-606n may be stored in the respective data store and/or in a metadata registry 614. The metadata registry 614 can be a separate data store, file, etc., that stores data store metadata information for each data store within the data storage system 602. The metadata registry 614 can include the same and/or different data store metadata information for each respective data store 606a-n. In some examples, the metadata registry 614 can include metadata determined for each data store 606a-n upon registering the data store to the data storage system 602. Registering a data store within the data storage system 602 can include providing information related to features supported by the data store, functionality of the data store, and other data store metadata that can be used for evaluation during the dry run 610. The metadata can include but is not limited to structural metadata (e.g., schema, indexes, data types, etc.), operational metadata (e.g., query execution plans, data store statistics, storage details, etc.), and administrative metadata (e.g., compliance rules, etc.).

Based on an evaluation of the data store metadata (e.g., from the metadata registry 614 and/or metadata 612a-n stored in each data store 606a-n), the query processing layer 608 identifies which data store best satisfies the constraint(s) 605 of the query 604. As an example, satisfaction of a feasibility constraint may be determined based features of a data store described in the metadata 612a-n. Such features can indicate whether the query can be executed on the data store. For example, if the intent is determined to be join, the query operations associated with executing a join (e.g., combining rows of a table) may not be executable on a vector data store, but may be executable on a relational database. As another example, the data store metadata can indicate a data store supports semantic searches. If the determined intent of the query 604 is to perform a semantic search, the dry run 610 can indicate the data store meets the feasibility criteria.

Additionally or alternatively, evaluation of freshness constraint satisfaction can include determining the freshness of data within a data store using the metadata and/or a watermark associated with the data store. The watermark may be determined as a timestamp of the oldest last successful updated data record within the data store. For example, the watermark associated with the data store can be the oldest watermark generated for a semantic object as described in FIG. 5. Additionally or alternatively, the freshness of data in the data store may be determined based on the last successful update timestamp to the data store as indicated in the data store metadata 612a-n. In some examples, an error status and/or error information may be included in the data store metadata 612a-n and/or in the metadata registry 614 that can be used to determine data freshness. In some examples, an age of the data in the data store may be determined by determining a difference between the current time and the watermark (e.g., last successful update timestamp) of the oldest records in the data store and/or a timestamp of the last successful update to the data store. In some examples, such as in the CDL syntax included above, the freshness constraint may be expressed as a max age of data, and satisfaction of the freshness constraint may be determined based on whether the age of data in each data store does not exceed the max age.

The query processing layer 608 may utilize one or more decision models to determine which data store best satisfies the constraint(s) 605. The decision models can include cost models, heuristics based rules, etc. that define how best the query 604 can be executed. As an example, the constraint(s) may include a freshness constraint, a feasibility constraint, and an execution time constraint. The dry run 610 may determine data stores 606a-b satisfy the feasibility constraint if the query intent can be executed on the data stores 606a-b. The dry run 610 may determine data stores 606a-b and data store 606d satisfy the freshness constraint as the max age of data within the data stores 606a-b and 606d do not exceed the max age indicated by the freshness constraint. Finally, the dry run 610 may determine data stores 606a-b and 606d-f satisfy the execution time constraint as the predicted execution time is less than the maximum acceptable query latency. In such instances, data store 606a or data store 606b may be selected as the optimal data store. The optimal data store may be selected based on additional system constraints (e.g. resource availability) and/or based on how well the data stores 606a-b satisfy the constraints (e.g., which data store has fresher data, which data store has a lower predicted execution time, etc.). Additionally or alternatively, the optimal data store may be selected by using a machine learning model trained on data from previous query executions (e.g., historical queries and associated constraints, metadata of corresponding data stores, etc.). The metadata and/or additional data store information can be provided to a machine learning model as an input and the optimal data store may be outputted by the model. For example, if data store 606a was previously selected as the optimal data store for a previously query with the same and/or similar constraints and the execution of the query on the data store 606a was successful, an output of the machine learning model may indicate data store 606a is the optimal data store.

Upon selecting the optimal data store, the query processing layer 608 can execute the query 604 on the optimal data store to obtain a query result 616. In some instances, the selected data store may not support execution of the query in the received language. For example, the query 604 may be a SQL query, but the optimal data store 606a may be a vector store. For such cases, the query 604 can be dynamically translated to the programming language that can be executed on data store 606a to obtain the query result 616. The query result 616 can then be provided to the user or other entity requesting the data (e.g., via the transaction layer). In some examples (e.g. as described with respect to FIGS. 1-2), the query result 616 can be provided to a components of a CDA system that can generate a contextual response including the query result 616 to provide to a user. Upon execution of the query, execution metadata including information about the selected optimal data store and execution analytics can be captured to enable adaptive learning and improvements on future routing decisions by the system.

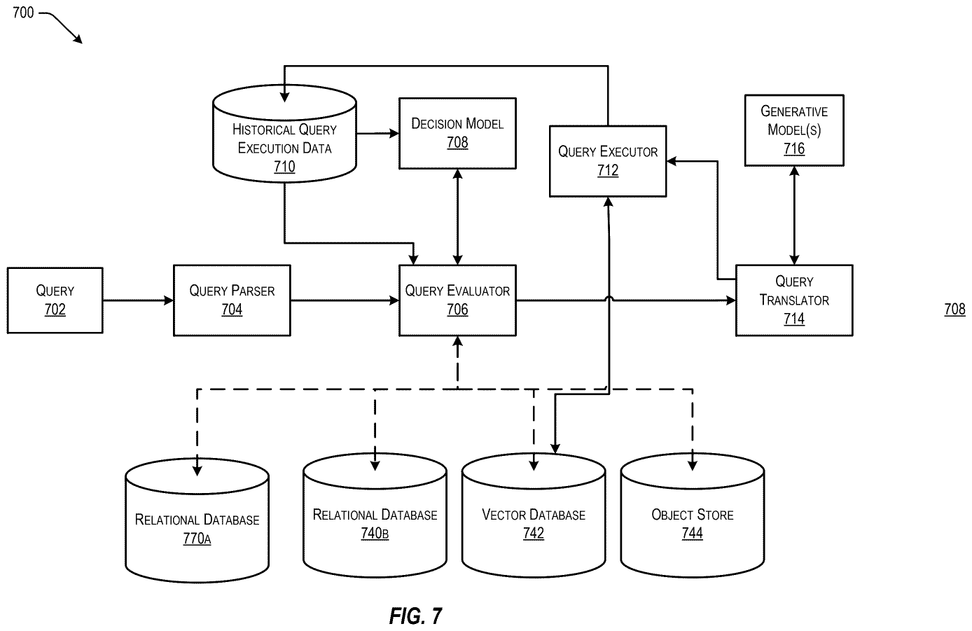

FIG. 7 is a block diagram illustrating data flow 700 for constraint-based query routing in a data storage system, in accordance with various embodiments. Various components are described with respect to components and processing of FIG. 6. The data flow 700 can be implemented in a heterogeneous data storage system (e.g., data storage system 602 of FIG. 6). Processing performed by the flow may be performed by the query processing layer described with respect to FIG. 6. For the purposes of illustration, the data storage system includes relational databases 740a-n, vector database 742, and object store 746, though this is not intended to be limiting and the data storage system can include additional, fewer, and/or different data stores than those depicted in FIG. 7.

A query 702 (e.g., query 604 of FIG. 6) is received at a data storage system. The query 702 can be a programming language query that may be executable on one or more of the data stores in the data system. In some examples, the query 702 can be received as a field of an API payload at an API endpoint of a transaction layer (e.g. transaction layer 306 of FIG. 3). As a particular example, the query 702 can be generated by a component of a digital assistant system (e.g., planner 204 of FIG. 2). The digital assistant system may receive a natural language utterance from an end user (e.g., a healthcare provider) requesting certain data of a patient and due to recent updates to the patient data, wants the most recent data possible. For such natural language utterances, the planner may generate the query 702 and indicate a freshness constraint with a certain staleness threshold should be satisfied when returning a query result.

The query 702 is provided to a query parser 704 to determine an intent of the query. The query parser 704 may be software, hardware, or a combination thereof configured to parse the query 702 and identify the intent of the query 702. As described above, the query parser 704 may identify key terms (e.g., as listed in Table 1) within the query 702 to identify the intent of the query. Additionally or alternatively, the query parser 704 may utilize a machine learning model trained on example queries and intents to determine an intent of the query 702.

In some implementations, the query parser 704 parses the query 702 and generates an abstract syntax tree (AST) representing a logical structure of the query 702. The query parser 704 can process the query 702 to identify key terms (e.g., tokens) that can be used to generate the AST. The key terms can include keywords, identifiers, operators, etc. The query parser 704 may identify the key terms by performing a lexical analysis (e.g., tokenization) of the query 702 to identify fundamental elements of the query 702. In some examples, the query 702 can be tokenized by an additional and/or alternative component (e.g., a tokenizer, lexer, etc.) and the tokenized query may be provided to the query parser 704 for parsing and intent determination. The query parser 704 can perform a syntax analysis of the tokenized query to generate the AST and identify the intent of the query 702 based on the logical structure reflected in the AST.

The intent identified by the query parser 704 is sent to a query evaluator 706 with the query 702 and the associated constraints. The query evaluator 706 performs a dry run of the various data stores to evaluate constraint satisfaction using metadata as described above with respect to FIG. 6. In some examples, the query evaluator 706 performs the dry run by using the metadata to compute a constraint satisfaction score for constraint and each data store. The constraint satisfaction score. In some examples, the constraint satisfaction score can be binary value (e.g., 0 or 1) indicating whether the data store satisfies the particular constraint. For example, for an intent determined to be a join, relational databases 740a-b that supports join queries may be assigned a constraint satisfaction score for feasibility of 1, while vector database 742 that does not support join queries may be assigned a 0. As another example, the intent may be identified as a semantic search. Relational database 740a may be a newer version of a particular database that supports semantic searches (e.g., as identified by database metadata) and may be assigned a feasibility constraint satisfaction score of 1, while relational database 704b may be an older version of the particular database the does not support semantic searches and may receive a constraint satisfaction score of 0. In some examples, the constraint satisfaction score may be determined based on how well the data store satisfies a particular constraint. For example, if vector database 742 includes data that is significantly fresher than the max age indicated by a freshness constraint, while relational database 740 has a data freshness that is equal to the max age, the vector database 742 may be assigned a higher freshness constraint satisfaction score than the relational database 740.

In some examples, the query evaluator 706 can select the optimal data store by computing a weighted sum using the constraint satisfaction scores. For example, the constraints may include priority values (e.g., as shown in the CDL syntax above). As an example, the constraints may be expressed using a CDL expression with priority operators (e.g., high, low, medium, etc.). Each priority operation may be associated with a corresponding numeric priority value (e.g., 1 for high, 0.25 for low, 0.5 for medium, etc.) that can be used to compute the weighted sum. Additionally or alternatively, the priority operators may be provided as numeric values. To calculate the weighted sum, each constraint satisfaction score can be multiplied by the corresponding priority value to generate weighted constraint satisfaction score. The sum of each weighted constraint satisfaction score can be computed to determine the weighted sum of constraint satisfaction scores. To determine whether the data store satisfies the constraints, the weighted sum can be compared to a constraint satisfaction threshold. If the weighted sum computed for a particular data store exceeds the constraint satisfaction threshold, the query evaluator 706 may determine the data store satisfies the one or more constraints. In some examples, the data store with the highest weighted sum may be selected as the optimal data store.

Additionally or alternatively, the query evaluator 706 may use a decision model 708 to identify the optimal data store. The decision model 708 may be a machine learning model trained on execution metadata 710 including information on historical query patterns. For example, the query evaluator 706 can provide the metadata for each data store, the identified intent, and the constraints as an input (e.g., a features array, tensors, etc.) to the decision model 708 to determine the optimal data store for query execution.

For the selected optimal data store, the query evaluator 706 determines whether the query 702 can be executed on the data store. In some examples, the query 702 may be executable on the optimal data store in the programming language it was received in. For example, if the query 702 is a SQL query and the optimal data store is relational database 740a, the query 702 may be executable on the relational database 740a. In such instances, the query evaluator 706 can provide the query 702 to a query executor 712 to execute the query 702 on the selected optimal data store.

In some instances, the optimal data store may not support queries in the programming language of the query 702. For example, if the optimal data is determined to be vector database 742 but the query 702 is an SQL query, the query evaluator 706 determines that the query should be rewritten to a query language executable on the vector database 742 (e.g., Vector DSL). The query evaluator 706 can provide the query to a query translator 714. The query translator 714 can be software, hardware, or combinations thereof configured to convert a query from one programming language to another programming language. In some examples, the query translator 714 may use rule based decision making to translate the query. For example, the query translator 714 may parse the query and use predefined mapping rules to translate the query into a translated query that can be executed on the optimal data store.

In some examples, the query translator 714 can be or can make use of one or more generative model(s) 716 (e.g., LLMs, LMMs) to translate the query 702. The generative model(s) 716 may be fine-tuned models for converting queries from one format and/or language to another (e.g., text-to-SQL, SQL-to-text, SQL-to-QDSL, etc.). For example, the generative model(s) 716 may be trained and/or fine-tuned using training data including instruction and response pairs showing an example prompt and an expected output. The training data instructions can include examples of queries in a first language (e.g., SQL) and the response in training data can include examples of translated queries in the second language (e.g., vector DSL). The query translator 714 can generate a prompt for the generative model 716 including the query 702 and instructions to translate the query to the intended programming language. The instructions can include one or more statements (e.g., natural language statements) indicating one or more requirements for the translated query to be executable on the optimal data store.

The generative model(s) 716 may use query metadata to generate the translated query. The query metadata may be extracted by the query parser 704 and may be provided to the generative model(s) 716 in a prompt. Query metadata can include fields accessed within the query 702, expressions used over the fields, query type, query intent, query conditions, and/or other similar properties of the query 702 that can be used to determine equivalent syntax and/or structure in the intended language. Additionally or alternatively, the query translator 714 can provide generative model(s) 716 with a readable format (e.g., string) version of an AST generated by the query parser 704. In some instances, the generative model(s) 716 are trained and/or prompted to extract relevant metadata from a provided AST. Additionally or alternatively, the generative model(s) 716 can be prompted to generate a translated query in the second programming language based on a query structure determined from the AST. For example, the generative model(s) 716 can be trained on a mapping of query structures between the first programming language and the second programming language and/or prompted with instructions for translating the query 702 using an AST. [0128] As a nonlimiting example, a SQL query can be as follows:
SELECT*FROM practitioner WHERE department=‘cardiology’;

In instances where the query evaluator determines the SQL query above should be routed to the vector database 742 for execution, the translated query in Query DSL can be as follows:

```
{
  “query”: {
    “term”: {
      “department”: “cardiology”
    }
  }
}
```

A query result generated upon executing the query 702 or the translated query can be obtained by the query executor. The query executor 712 can collect execution metadata based on the execution of the query. Execution metadata can include but is not limited to query response time, freshness level of the selected optimal data store, and the feasibility status of the optimal data store. Additionally or alternatively, the execution metadata can include a user satisfaction score for the execution. The execution metadata collected during query execution included in historical query execution data 710 used by the decision model 708 to evaluate queries during a dry run. For example, in some instances, in examples where the decision model 708 and/or query evaluator 706 compute a weighted sum, the historical query execution data 710 including the execution metadata can be used to compute an additional and/or alternative weight for each constraint satisfaction score. Additionally or alternatively, the decision model 708 may be periodically retrained using the historical query execution data 710 to improve model performance on query routing for combinations of queries, intents, and constraints that have previously been received. The retrained decision model 708 may be applied to new queries to ensure the system accurately and adaptively learns from previous query routing performance. Additionally or alternatively, the query evaluator 706 may determine whether a newly received query is that same and/or similar to a previously received query (e.g., query 702). In such instances, the query evaluator 706 may retrieve execution metadata and/or other historical query execution data 710 to determine whether the new query can be routed to the previously selected optimal data store. Additionally or alternatively, the query evaluator 706 may determine based on the historical query execution data 710 that a particular data store cannot meet the constraints (e.g., feasibility) of a particular query and may perform the dry run evaluation on a subset of the data stores that excludes the particular data store.

### Illustrative Methods

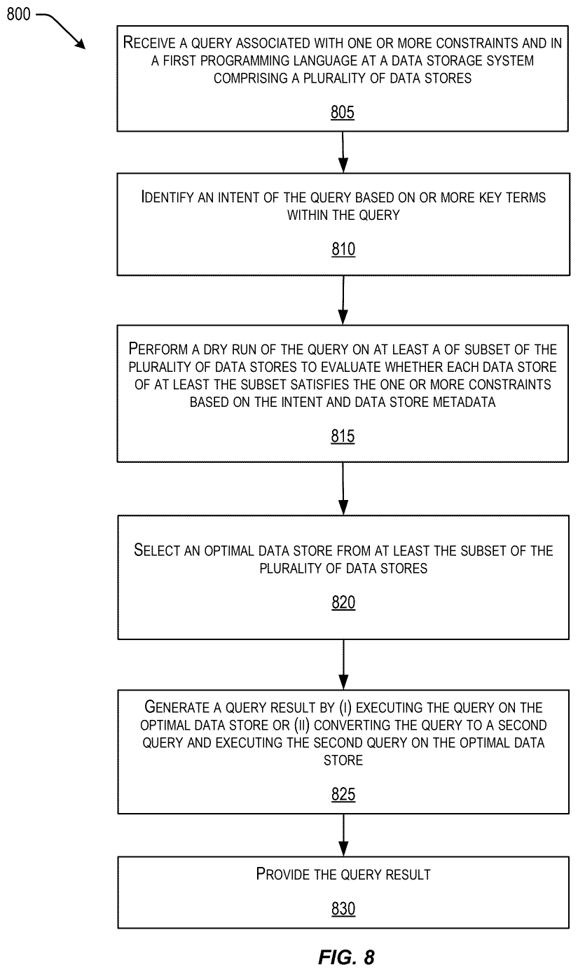

FIG. 8 is a flowchart of a process constraint-driven query routing in data systems with disparate data stores, in accordance with various embodiments. The processing depicted in FIG. 8 may be implemented in software (e.g., code, instructions, program) executed by one or more processing units (e.g., processors, cores) of the respective systems, hardware, or combinations thereof. The software may be stored on a non-transitory storage medium (e.g., on a memory device). The process presented in FIG. 8 and described below is intended to be illustrative and non-limiting. Although FIG. 8 illustrates the various processing steps occurring in a particular sequence or order, this is not intended to be limiting. In certain alternative embodiments, the steps may be performed in some different order or some steps may also be performed at least partially in parallel. In certain embodiments, the processing depicted in FIG. 8 may be performed by one or more of the components, computing devices, services, or the like, such as a data storage system, semantic index, etc., illustrated and described with respect to FIGS. 1-7 and FIGS. 9-13.

At step 805, a query (e.g., query 604 of FIG. 6) is received at a data storage system (e.g., data storage system 602 of FIG. 6). The data storage system can include a plurality of data stores (e.g., data stores 606a-n of FIG. 6). The query can be associated with one or more constraints. The one or more constraints can include a freshness constraint, a feasibility constraint, an execution time constraint, or any combination thereof. The query may be in a first programming language. In some examples, the constraints can be expressed in a constraint definition language (CDL). The expression of the one or more constraints in the CDL can indicate a logical precedence of the one or more constraints. In some examples, the logical precedence may be indicated using operators such as AND, and OR. In some examples, a priority level of the one or more constraints may be indicated.

At step 810, an intent of the query is identified from a set of intents based on one or more key terms within the query. The set of intents can include, but is not limited to, a point query, a filter query, a join query, an aggregate query, a subquery, or any combination thereof. In some examples, the intent of the query can be determined by using the key term of the query to determine a query structure. For example, the query can be tokenized and parsed (e.g., by query parser 704 of FIG. 7) to generate a abstract syntax tree (AST) that can be used to determine the intent based on query language rules.

At step 815, a dry run (e.g., dry run 610) of the query is performed on at least a subset of the plurality of data stores. The dry run can be performed to evaluate, for each data store of at least the subset, whether the data store satisfies the one or more constraints based on the intent and data store metadata (e.g., metadata 612a-n of FIG. 6, metadata from metadata registry 614 of FIG. 6). The data store metadata can include data store information associated with each data store of at least the subset. The data store information can include information associated with (i) a data model of the data store, (ii) a schema of the data store, (iii) a set of features of the data store, or (iv) any combination thereof.

At step 820, an optimal data store is selected from at least the subset of the plurality of data stores and based on the dry run. In some examples, the one or more constraints include the freshness constraint and performing the dry run includes computing a watermark indicating freshness of data within the data store based on the data store metadata for each data store of at least the subset. A comparison of the watermark and an expected freshness indicated by the freshness constraint can be performed to determine whether the data store satisfies the freshness constraint.

In some examples, the one or more constraints include the feasibility constraint and performing the dry run includes, for each data store of at least the subset, determining whether the query can be executed the data store based on the intent and one or more features of the data store. The one or more features can be determined based on the data store metadata.

In some examples, performing the dry run includes, for each data store of at least the subset, determining a constraint satisfaction score for each constraint of the one or more constraints based on the intent and the data store metadata. A weighted sum of the constraint satisfaction scores can be computed and performing the dry can include determining whether the weighted sum matches or exceeds a constraint satisfaction threshold. If the weighted sum matches or exceeds the constraint satisfaction threshold, the data store may satisfy the one or more constraints. If the weighted sum does not match or exceed the constraint satisfaction threshold, the data store may not satisfy the one or more constraints.

In some examples, selecting the optimal data store can include applying a decision model (e.g., decision model 708 of FIG. 7). The decision model may be a machine learning decision model trained on historical query execution patterns (e.g., historical query execution data 710 of FIG. 7).

At step 825, a query result for the query is generated. Generating the query result can include determining whether the query in the first programming language can be executed on the optimal data store. In response to determining the query in the first programming language can be executed on the optimal data store, the query can be executed on the optimal data store to obtain the query result. In response to determining the query in the first programming language cannot be executed on the optimal data store, the query can be converted to a second query in a second programming language (e.g., by query translator 714 of FIG. 7) that can be executed on the optimal data store. The second query can be executed on the optimal data store to obtain the query result.

In some examples, a prompt including the query and one or more instructions to translate the query from the first programming language to the second programming language can be provided to a generative model (e.g., generative model(s) 716 of FIG. 7). The second query corresponding to the second programming language may be received from the generated model. The generative model can generate the second query based on the prompt and the second programming language can be a programming language that can be executed on the optimal data store.

In some examples, execution metadata associated with the execution of the query or the execution of the second query can be collected. The execution metadata may include a query response time, freshness level, feasibility status, user satisfaction, or any combination thereof. A subsequent query may be received associated with at least one of the one or more constraints. The optimal data store may be selected based on the execution metadata and historical query execution data.

At step 830, the query result is provided. In some examples, the query result may be provided to an entity (e.g., a component of a CDA system described with respect to FIGS. 1-2) that generates a response (e.g., a natural language response) including the query result and provides the response to a user.

### Examples of Architectures for Implementing Cloud Infrastructures

As noted above, infrastructure as a service (IaaS) is one particular type of cloud computing. IaaS can be configured to provide virtualized computing resources over a public network (e.g., the Internet). In an IaaS model, a cloud computing provider can host the infrastructure components (e.g., servers, storage devices, network nodes (e.g., hardware), deployment software, platform virtualization (e.g., a hypervisor layer), or the like). In some cases, an IaaS provider may also supply a variety of services to accompany those infrastructure components (example services include billing software, monitoring software, logging software, load balancing software, clustering software, etc.). Thus, as these services may be policy-driven, IaaS users may be able to implement policies to drive load balancing to maintain application availability and performance.

In some instances, IaaS customers may access resources and services through a wide area network (WAN), such as the Internet, and can use the cloud provider's services to install the remaining elements of an application stack. For example, the user can log in to the IaaS platform to create virtual machines (VMs), install operating systems (OSs) on each VM, deploy middleware such as databases, create storage buckets for workloads and backups, and even install enterprise software into that VM. Customers can then use the provider's services to perform various functions, including balancing network traffic, troubleshooting application issues, monitoring performance, managing disaster recovery, etc.

In most cases, a cloud computing model will require the participation of a cloud provider. The cloud provider may, but need not be, a third-party service that specializes in providing (e.g., offering, renting, selling) IaaS. An entity might also opt to deploy a private cloud, becoming its own provider of infrastructure services.

In some examples, IaaS deployment is the process of putting a new application, or a new version of an application, onto a prepared application server or the like. It may also include the process of preparing the server (e.g., installing libraries, daemons, etc.). This is often managed by the cloud provider, below the hypervisor layer (e.g., the servers, storage, network hardware, and virtualization). Thus, the customer may be responsible for handling (OS), middleware, and/or application deployment (e.g., on self-service virtual machines (e.g., that can be spun up on demand)) or the like.

In some examples, IaaS provisioning may refer to acquiring computers or virtual hosts for use, and even installing needed libraries or services on them. In most cases, deployment does not include provisioning, and the provisioning may need to be performed first.

In some cases, there are two different challenges for IaaS provisioning. First, there is the initial challenge of provisioning the initial set of infrastructure before anything is running. Second, there is the challenge of evolving the existing infrastructure (e.g., adding new services, changing services, removing services, etc.) once everything has been provisioned. In some cases, these two challenges may be addressed by enabling the configuration of the infrastructure to be defined declaratively. In other words, the infrastructure (e.g., what components are needed and how they interact) can be defined by one or more configuration files. Thus, the overall topology of the infrastructure (e.g., what resources depend on which, and how they each work together) can be described declaratively. In some instances, once the topology is defined, a workflow can be generated that creates and/or manages the different components described in the configuration files.

In some examples, an infrastructure may have many interconnected elements. For example, there may be one or more virtual private clouds (VPCs) (e.g., a potentially on-demand pool of configurable and/or shared computing resources), also known as a core network. In some examples, there may also be one or more inbound/outbound traffic group rules provisioned to define how the inbound and/or outbound traffic of the network will be set up and one or more virtual machines (VMs). Other infrastructure elements may also be provisioned, such as a load balancer, a database, or the like. As more and more infrastructure elements are desired and/or added, the infrastructure may incrementally evolve.

In some instances, continuous deployment techniques may be employed to enable deployment of infrastructure code across various virtual computing environments. Additionally, the described techniques can enable infrastructure management within these environments. In some examples, service teams can write code that is desired to be deployed to one or more, but often many, different production environments (e.g., across various different geographic locations, sometimes spanning the entire world). However, in some examples, the infrastructure on which the code will be deployed must first be set up. In some instances, the provisioning can be done manually, a provisioning tool may be utilized to provision the resources, and/or deployment tools may be utilized to deploy the code once the infrastructure is provisioned.

FIG. 9 is a block diagram 900 illustrating an example pattern of an IaaS architecture, according to at least one embodiment. Service operators 902 can be communicatively coupled to a secure host tenancy 904 that can include a virtual cloud network (VCN) 906 and a secure host subnet 908. In some examples, the service operators 902 may be using one or more client computing devices, which may be portable handheld devices (e.g., an iPhone®, cellular telephone, an iPad®, computing tablet, a personal digital assistant (PDA)) or wearable devices (e.g., a Google Glass® head mounted display), running software such as Microsoft Windows Mobile®, and/or a variety of mobile operating systems such as iOS, Windows Phone, Android, BlackBerry 8, Palm OS, and the like, and being Internet, e-mail, short message service (SMS), Blackberry®, or other communication protocol enabled. Alternatively, the client computing devices can be general purpose personal computers including, by way of example, personal computers and/or laptop computers running various versions of Microsoft Windows®, Apple Macintosh®, and/or Linux operating systems. The client computing devices can be workstation computers running any of a variety of commercially-available UNIX® or UNIX-like operating systems, including without limitation the variety of GNU/Linux operating systems, such as for example, Google Chrome OS. Alternatively, or in addition, client computing devices may be any other electronic device, such as a thin-client computer, an Internet-enabled gaming system (e.g., a Microsoft Xbox gaming console with or without a Kinect® gesture input device), and/or a personal messaging device, capable of communicating over a network that can access the VCN 906 and/or the Internet.

The VCN 906 can include a local peering gateway (LPG) 910 that can be communicatively coupled to a secure shell (SSH) VCN 912 via an LPG 910 contained in the SSH VCN 912. The SSH VCN 912 can include an SSH subnet 914, and the SSH VCN 912 can be communicatively coupled to a control plane VCN 916 via the LPG 910 contained in the control plane VCN 916. Also, the SSH VCN 912 can be communicatively coupled to a data plane VCN 918 via an LPG 910. The control plane VCN 916 and the data plane VCN 918 can be contained in a service tenancy 919 that can be owned and/or operated by the IaaS provider.

The control plane VCN 916 can include a control plane demilitarized zone (DMZ) tier 920 that acts as a perimeter network (e.g., portions of a corporate network between the corporate intranet and external networks). The DMZ-based servers may have restricted responsibilities and help keep breaches contained. Additionally, the DMZ tier 920 can include one or more load balancer (LB) subnet(s) 922, a control plane app tier 924 that can include app subnet(s) 926, a control plane data tier 928 that can include database (DB) subnet(s) 930 (e.g., frontend DB subnet(s) and/or backend DB subnet(s)). The LB subnet(s) 922 contained in the control plane DMZ tier 920 can be communicatively coupled to the app subnet(s) 926 contained in the control plane app tier 924 and an Internet gateway 934 that can be contained in the control plane VCN 916, and the app subnet(s) 926 can be communicatively coupled to the DB subnet(s) 930 contained in the control plane data tier 928 and a service gateway 936 and a network address translation (NAT) gateway 938. The control plane VCN 916 can include the service gateway 936 and the NAT gateway 938.

The control plane VCN 916 can include a data plane mirror app tier 940 that can include app subnet(s) 926. The app subnet(s) 926 contained in the data plane mirror app tier 940 can include a virtual network interface controller (VNIC) 942 that can execute a compute instance 944. The compute instance 944 can communicatively couple the app subnet(s) 926 of the data plane mirror app tier 940 to app subnet(s) 926 that can be contained in a data plane app tier 946.

The data plane VCN 918 can include the data plane app tier 946, a data plane DMZ tier 948, and a data plane data tier 950. The data plane DMZ tier 948 can include LB subnet(s) 922 that can be communicatively coupled to the app subnet(s) 926 of the data plane app tier 946 and the Internet gateway 934 of the data plane VCN 918. The app subnet(s) 926 can be communicatively coupled to the service gateway 936 of the data plane VCN 918 and the NAT gateway 938 of the data plane VCN 918. The data plane data tier 950 can also include the DB subnet(s) 930 that can be communicatively coupled to the app subnet(s) 926 of the data plane app tier 946.

The Internet gateway 934 of the control plane VCN 916 and of the data plane VCN 918 can be communicatively coupled to a metadata management service 952 that can be communicatively coupled to public Internet 954. Public Internet 954 can be communicatively coupled to the NAT gateway 938 of the control plane VCN 916 and of the data plane VCN 918. The service gateway 936 of the control plane VCN 916 and of the data plane VCN 918 can be communicatively coupled to cloud services 956.

In some examples, the service gateway 936 of the control plane VCN 916 or of the data plane VCN 918 can make application programming interface (API) calls to cloud services 956 without going through public Internet 954. The API calls to cloud services 956 from the service gateway 936 can be one-way: the service gateway 936 can make API calls to cloud services 956, and cloud services 956 can send requested data to the service gateway 936. But, cloud services 956 may not initiate API calls to the service gateway 936.

In some examples, the secure host tenancy 904 can be directly connected to the service tenancy 919, which may be otherwise isolated. The secure host subnet 908 can communicate with the SSH subnet 914 through an LPG 910 that may enable two-way communication over an otherwise isolated system. Connecting the secure host subnet 908 to the SSH subnet 914 may give the secure host subnet 908 access to other entities within the service tenancy 919.

The control plane VCN 916 may allow users of the service tenancy 919 to set up or otherwise provision desired resources. Desired resources provisioned in the control plane VCN 916 may be deployed or otherwise used in the data plane VCN 918. In some examples, the control plane VCN 916 can be isolated from the data plane VCN 918, and the data plane mirror app tier 940 of the control plane VCN 916 can communicate with the data plane app tier 946 of the data plane VCN 918 via VNICs 942 that can be contained in the data plane mirror app tier 940 and the data plane app tier 946.

In some examples, users of the system, or customers, can make requests, for example create, read, update, or delete (CRUD) operations, through public Internet 954 that can communicate the requests to the metadata management service 952. The metadata management service 952 can communicate the request to the control plane VCN 916 through the Internet gateway 934. The request can be received by the LB subnet(s) 922 contained in the control plane DMZ tier 920. The LB subnet(s) 922 may determine that the request is valid, and in response to this determination, the LB subnet(s) 922 can transmit the request to app subnet(s) 926 contained in the control plane app tier 924. If the request is validated and requires a call to public Internet 954, the call to public Internet 954 may be transmitted to the NAT gateway 938 that can make the call to public Internet 954. Metadata that may be desired to be stored by the request can be stored in the DB subnet(s) 930.

In some examples, the data plane mirror app tier 940 can facilitate direct communication between the control plane VCN 916 and the data plane VCN 918. For example, changes, updates, or other suitable modifications to configuration may be desired to be applied to the resources contained in the data plane VCN 918. Via a VNIC 942, the control plane VCN 916 can directly communicate with, and can thereby execute the changes, updates, or other suitable modifications to configuration to, resources contained in the data plane VCN 918.

In some embodiments, the control plane VCN 916 and the data plane VCN 918 can be contained in the service tenancy 919. In this case, the user, or the customer, of the system may not own or operate either the control plane VCN 916 or the data plane VCN 918. Instead, the IaaS provider may own or operate the control plane VCN 916 and the data plane VCN 918, both of which may be contained in the service tenancy 919. This embodiment can enable isolation of networks that may prevent users or customers from interacting with other users', or other customers', resources. Also, this embodiment may allow users or customers of the system to store databases privately without needing to rely on public Internet 954, which may not have a desired level of threat prevention, for storage.

In other embodiments, the LB subnet(s) 922 contained in the control plane VCN 916 can be configured to receive a signal from the service gateway 936. In this embodiment, the control plane VCN 916 and the data plane VCN 918 may be configured to be called by a customer of the IaaS provider without calling public Internet 954. Customers of the IaaS provider may desire this embodiment since database(s) that the customers use may be controlled by the IaaS provider and may be stored on the service tenancy 919, which may be isolated from public Internet 954.

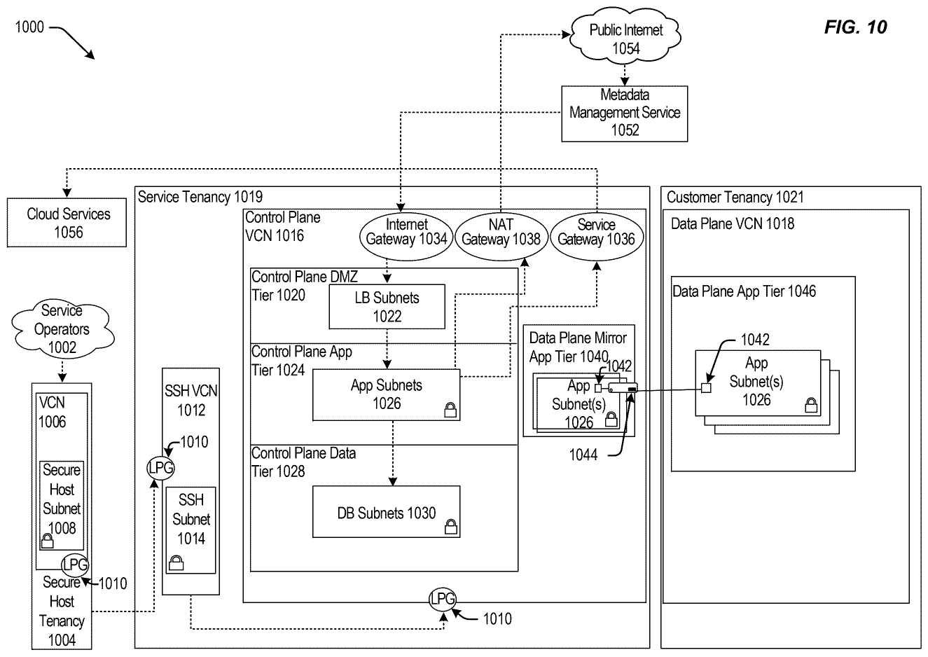

FIG. 10 is a block diagram 1000 illustrating another example pattern of an IaaS architecture, according to at least one embodiment. Service operators 1002 (e.g., service operators 902 of FIG. 9) can be communicatively coupled to a secure host tenancy 1004 (e.g., the secure host tenancy 904 of FIG. 9) that can include a virtual cloud network (VCN) 1006 (e.g., the VCN 906 of FIG. 9) and a secure host subnet 1008 (e.g., the secure host subnet 908 of FIG. 9). The VCN 1006 can include a local peering gateway (LPG) 1010 (e.g., the LPG 910 of FIG. 9) that can be communicatively coupled to a secure shell (SSH) VCN 1012 (e.g., the SSH VCN 912 of FIG. 9) via an LPG 910 contained in the SSH VCN 1012. The SSH VCN 1012 can include an SSH subnet 1014 (e.g., the SSH subnet 914 of FIG. 9), and the SSH VCN 1012 can be communicatively coupled to a control plane VCN 1016 (e.g., the control plane VCN 916 of FIG. 9) via an LPG 1010 contained in the control plane VCN 1016. The control plane VCN 1016 can be contained in a service tenancy 1019 (e.g., the service tenancy 919 of FIG. 9), and the data plane VCN 1018 (e.g., the data plane VCN 918 of FIG. 9) can be contained in a customer tenancy 1021 that may be owned or operated by users, or customers, of the system.

The control plane VCN 1016 can include a control plane DMZ tier 1020 (e.g., the control plane DMZ tier 920 of FIG. 9) that can include LB subnet(s) 1022 (e.g., LB subnet(s) 922 of FIG. 9), a control plane app tier 1024 (e.g., the control plane app tier 924 of FIG. 9) that can include app subnet(s) 1026 (e.g., app subnet(s) 926 of FIG. 9), a control plane data tier 1028 (e.g., the control plane data tier 928 of FIG. 9) that can include database (DB) subnet(s) 1030 (e.g., similar to DB subnet(s) 930 of FIG. 9). The LB subnet(s) 1022 contained in the control plane DMZ tier 1020 can be communicatively coupled to the app subnet(s) 1026 contained in the control plane app tier 1024 and an Internet gateway 1034 (e.g., the Internet gateway 934 of FIG. 9) that can be contained in the control plane VCN 1016, and the app subnet(s) 1026 can be communicatively coupled to the DB subnet(s) 1030 contained in the control plane data tier 1028 and a service gateway 1036 (e.g., the service gateway 936 of FIG. 9) and a network address translation (NAT) gateway 1038 (e.g., the NAT gateway 938 of FIG. 9). The control plane VCN 1016 can include the service gateway 1036 and the NAT gateway 1038.

The control plane VCN 1016 can include a data plane mirror app tier 1040 (e.g., the data plane mirror app tier 940 of FIG. 9) that can include app subnet(s) 1026. The app subnet(s) 1026 contained in the data plane mirror app tier 1040 can include a virtual network interface controller (VNIC) 1042 (e.g., the VNIC of 942) that can execute a compute instance 1044 (e.g., similar to the compute instance 944 of FIG. 9). The compute instance 1044 can facilitate communication between the app subnet(s) 1026 of the data plane mirror app tier 1040 and the app subnet(s) 1026 that can be contained in a data plane app tier 1046 (e.g., the data plane app tier 946 of FIG. 9) via the VNIC 1042 contained in the data plane mirror app tier 1040 and the VNIC 1042 contained in the data plane app tier 1046.

The Internet gateway 1034 contained in the control plane VCN 1016 can be communicatively coupled to a metadata management service 1052 (e.g., the metadata management service 952 of FIG. 9) that can be communicatively coupled to public Internet 1054 (e.g., public Internet 954 of FIG. 9). Public Internet 1054 can be communicatively coupled to the NAT gateway 1038 contained in the control plane VCN 1016. The service gateway 1036 contained in the control plane VCN 1016 can be communicatively coupled to cloud services 1056 (e.g., cloud services 956 of FIG. 9).

In some examples, the data plane VCN 1018 can be contained in the customer tenancy 1021. In this case, the IaaS provider may provide the control plane VCN 1016 for each customer, and the IaaS provider may, for each customer, set up a unique compute instance 1044 that is contained in the service tenancy 1019. Each compute instance 1044 may allow communication between the control plane VCN 1016, contained in the service tenancy 1019, and the data plane VCN 1018 that is contained in the customer tenancy 1021. The compute instance 1044 may allow resources, that are provisioned in the control plane VCN 1016 that is contained in the service tenancy 1019, to be deployed or otherwise used in the data plane VCN 1018 that is contained in the customer tenancy 1021.

In other examples, the customer of the IaaS provider may have databases that live in the customer tenancy 1021. In this example, the control plane VCN 1016 can include the data plane mirror app tier 1040 that can include app subnet(s) 1026. The data plane mirror app tier 1040 can reside in the data plane VCN 1018, but the data plane mirror app tier 1040 may not live in the data plane VCN 1018. That is, the data plane mirror app tier 1040 may have access to the customer tenancy 1021, but the data plane mirror app tier 1040 may not exist in the data plane VCN 1018 or be owned or operated by the customer of the IaaS provider. The data plane mirror app tier 1040 may be configured to make calls to the data plane VCN 1018 but may not be configured to make calls to any entity contained in the control plane VCN 1016. The customer may desire to deploy or otherwise use resources in the data plane VCN 1018 that are provisioned in the control plane VCN 1016, and the data plane mirror app tier 1040 can facilitate the desired deployment, or other usage of resources, of the customer.

In some embodiments, the customer of the IaaS provider can apply filters to the data plane VCN 1018. In this embodiment, the customer can determine what the data plane VCN 1018 can access, and the customer may restrict access to public Internet 1054 from the data plane VCN 1018. The IaaS provider may not be able to apply filters or otherwise control access of the data plane VCN 1018 to any outside networks or databases. Applying filters and controls by the customer onto the data plane VCN 1018, contained in the customer tenancy 1021, can help isolate the data plane VCN 1018 from other customers and from public Internet 1054.

In some embodiments, cloud services 1056 can be called by the service gateway 1036 to access services that may not exist on public Internet 1054, on the control plane VCN 1016, or on the data plane VCN 1018. The connection between cloud services 1056 and the control plane VCN 1016 or the data plane VCN 1018 may not be live or continuous. Cloud services 1056 may exist on a different network owned or operated by the IaaS provider. Cloud services 1056 may be configured to receive calls from the service gateway 1036 and may be configured to not receive calls from public Internet 1054. Some cloud services 1056 may be isolated from other cloud services 1056, and the control plane VCN 1016 may be isolated from cloud services 1056 that may not be in the same region as the control plane VCN 1016. For example, the control plane VCN 1016 may be located in “Region 1,” and cloud service “Deployment 9,” may be located in Region 1 and in “Region 2.” If a call to Deployment 9 is made by the service gateway 1036 contained in the control plane VCN 1016 located in Region 1, the call may be transmitted to Deployment 9 in Region 1. In this example, the control plane VCN 1016, or Deployment 9 in Region 1, may not be communicatively coupled to, or otherwise in communication with, Deployment 9 in Region 2.

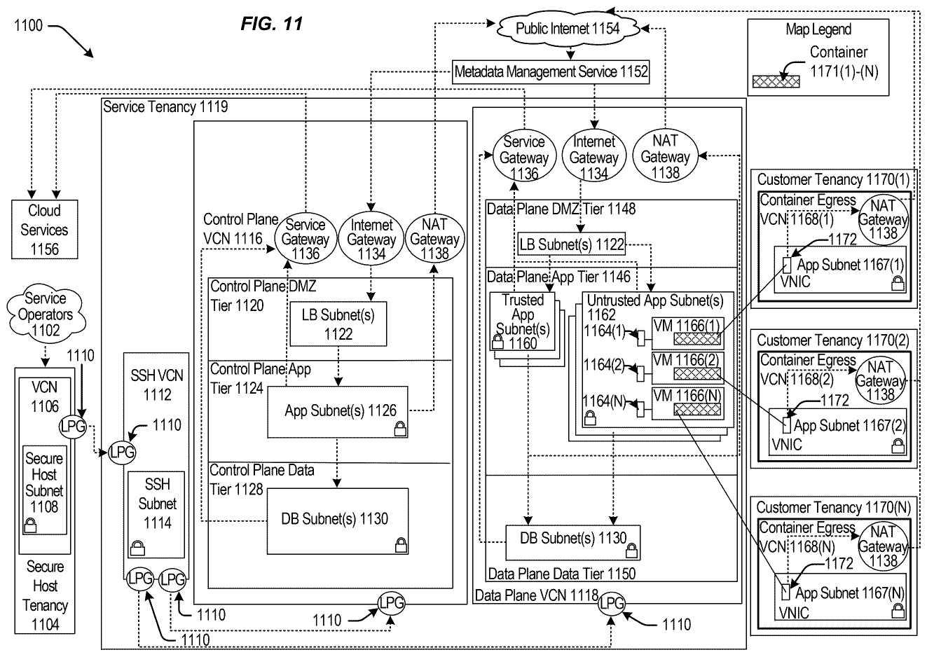

FIG. 11 is a block diagram 1100 illustrating another example pattern of an IaaS architecture, according to at least one embodiment. Service operators 1102 (e.g., service operators 902 of FIG. 9) can be communicatively coupled to a secure host tenancy 1104 (e.g., the secure host tenancy 904 of FIG. 9) that can include a virtual cloud network (VCN) 1106 (e.g., the VCN 906 of FIG. 9) and a secure host subnet 1108 (e.g., the secure host subnet 908 of FIG. 9). The VCN 1106 can include an LPG 1110 (e.g., the LPG 910 of FIG. 9) that can be communicatively coupled to an SSH VCN 1112 (e.g., the SSH VCN 912 of FIG. 9) via an LPG 1110 contained in the SSH VCN 1112. The SSH VCN 1112 can include an SSH subnet 1114 (e.g., the SSH subnet 914 of FIG. 9), and the SSH VCN 1112 can be communicatively coupled to a control plane VCN 1116 (e.g., the control plane VCN 916 of FIG. 9) via an LPG 1110 contained in the control plane VCN 1116 and to a data plane VCN 1118 (e.g., the data plane 918 of FIG. 9) via an LPG 1110 contained in the data plane VCN 1118. The control plane VCN 1116 and the data plane VCN 1118 can be contained in a service tenancy 1119 (e.g., the service tenancy 919 of FIG. 9).

The control plane VCN 1116 can include a control plane DMZ tier 1120 (e.g., the control plane DMZ tier 920 of FIG. 9) that can include load balancer (LB) subnet(s) 1122 (e.g., LB subnet(s) 922 of FIG. 9), a control plane app tier 1124 (e.g., the control plane app tier 924 of FIG. 9) that can include app subnet(s) 1126 (e.g., similar to app subnet(s) 926 of FIG. 9), a control plane data tier 1128 (e.g., the control plane data tier 928 of FIG. 9) that can include DB subnet(s) 1130. The LB subnet(s) 1122 contained in the control plane DMZ tier 1120 can be communicatively coupled to the app subnet(s) 1126 contained in the control plane app tier 1124 and to an Internet gateway 1134 (e.g., the Internet gateway 934 of FIG. 9) that can be contained in the control plane VCN 1116, and the app subnet(s) 1126 can be communicatively coupled to the DB subnet(s) 1130 contained in the control plane data tier 1128 and to a service gateway 1136 (e.g., the service gateway of FIG. 9) and a network address translation (NAT) gateway 1138 (e.g., the NAT gateway 938 of FIG. 9). The control plane VCN 1116 can include the service gateway 1136 and the NAT gateway 1138.

The data plane VCN 1118 can include a data plane app tier 1146 (e.g., the data plane app tier 946 of FIG. 9), a data plane DMZ tier 1148 (e.g., the data plane DMZ tier 948 of FIG. 9), and a data plane data tier 1150 (e.g., the data plane data tier 950 of FIG. 9). The data plane DMZ tier 1148 can include LB subnet(s) 1122 that can be communicatively coupled to trusted app subnet(s) 1160 and untrusted app subnet(s) 1162 of the data plane app tier 1146 and the Internet gateway 1134 contained in the data plane VCN 1118. The trusted app subnet(s) 1160 can be communicatively coupled to the service gateway 1136 contained in the data plane VCN 1118, the NAT gateway 1138 contained in the data plane VCN 1118, and DB subnet(s) 1130 contained in the data plane data tier 1150. The untrusted app subnet(s) 1162 can be communicatively coupled to the service gateway 1136 contained in the data plane VCN 1118 and DB subnet(s) 1130 contained in the data plane data tier 1150. The data plane data tier 1150 can include DB subnet(s) 1130 that can be communicatively coupled to the service gateway 1136 contained in the data plane VCN 1118.

The untrusted app subnet(s) 1162 can include one or more primary VNICs 1164(1)-(N) that can be communicatively coupled to tenant virtual machines (VMs) 1166(1)-(N). Each tenant VM 1166(1)-(N) can be communicatively coupled to a respective app subnet 1167(1)-(N) that can be contained in respective container egress VCNs 1168(1)-(N) that can be contained in respective customer tenancies 1170(1)-(N). Respective secondary VNICs 1172(1)-(N) can facilitate communication between the untrusted app subnet(s) 1162 contained in the data plane VCN 1118 and the app subnet contained in the container egress VCNs 1168(1)-(N). Each container egress VCNs 1168(1)-(N) can include a NAT gateway 1138 that can be communicatively coupled to public Internet 1154 (e.g., public Internet 954 of FIG. 9).

The Internet gateway 1134 contained in the control plane VCN 1116 and contained in the data plane VCN 1118 can be communicatively coupled to a metadata management service 1152 (e.g., the metadata management system 952 of FIG. 9) that can be communicatively coupled to public Internet 1154. Public Internet 1154 can be communicatively coupled to the NAT gateway 1138 contained in the control plane VCN 1116 and contained in the data plane VCN 1118. The service gateway 1136 contained in the control plane VCN 1116 and contained in the data plane VCN 1118 can be communicatively coupled to cloud services 1156.

In some embodiments, the data plane VCN 1118 can be integrated with customer tenancies 1170. This integration can be useful or desirable for customers of the IaaS provider in some cases such as a case that may desire support when executing code. The customer may provide code to run that may be destructive, may communicate with other customer resources, or may otherwise cause undesirable effects. In response to this, the IaaS provider may determine whether to run code given to the IaaS provider by the customer.

In some examples, the customer of the IaaS provider may grant temporary network access to the IaaS provider and request a function to be attached to the data plane app tier 1146. Code to run the function may be executed in the VMs 1166(1)-(N), and the code may not be configured to run anywhere else on the data plane VCN 1118. Each VM 1166(1)-(N) may be connected to one customer tenancy 1170. Respective containers 1171(1)-(N) contained in the VMs 1166(1)-(N) may be configured to run the code. In this case, there can be a dual isolation (e.g., the containers 1171(1)-(N) running code, where the containers 1171(1)-(N) may be contained in at least the VM 1166(1)-(N) that are contained in the untrusted app subnet(s) 1162), which may help prevent incorrect or otherwise undesirable code from damaging the network of the IaaS provider or from damaging a network of a different customer. The containers 1171(1)-(N) may be communicatively coupled to the customer tenancy 1170 and may be configured to transmit or receive data from the customer tenancy 1170. The containers 1171(1)-(N) may not be configured to transmit or receive data from any other entity in the data plane VCN 1118. Upon completion of running the code, the IaaS provider may kill or otherwise dispose of the containers 1171(1)-(N).

In some embodiments, the trusted app subnet(s) 1160 may run code that may be owned or operated by the IaaS provider. In this embodiment, the trusted app subnet(s) 1160 may be communicatively coupled to the DB subnet(s) 1130 and be configured to execute CRUD operations in the DB subnet(s) 1130. The untrusted app subnet(s) 1162 may be communicatively coupled to the DB subnet(s) 1130, but in this embodiment, the untrusted app subnet(s) may be configured to execute read operations in the DB subnet(s) 1130. The containers 1171(1)-(N) that can be contained in the VM 1166(1)-(N) of each customer and that may run code from the customer may not be communicatively coupled with the DB subnet(s) 1130.

In other embodiments, the control plane VCN 1116 and the data plane VCN 1118 may not be directly communicatively coupled. In this embodiment, there may be no direct communication between the control plane VCN 1116 and the data plane VCN 1118. However, communication can occur indirectly through at least one method. An LPG 1110 may be established by the IaaS provider that can facilitate communication between the control plane VCN 1116 and the data plane VCN 1118. In another example, the control plane VCN 1116 or the data plane VCN 1118 can make a call to cloud services 1156 via the service gateway 1136. For example, a call to cloud services 1156 from the control plane VCN 1116 can include a request for a service that can communicate with the data plane VCN 1118.

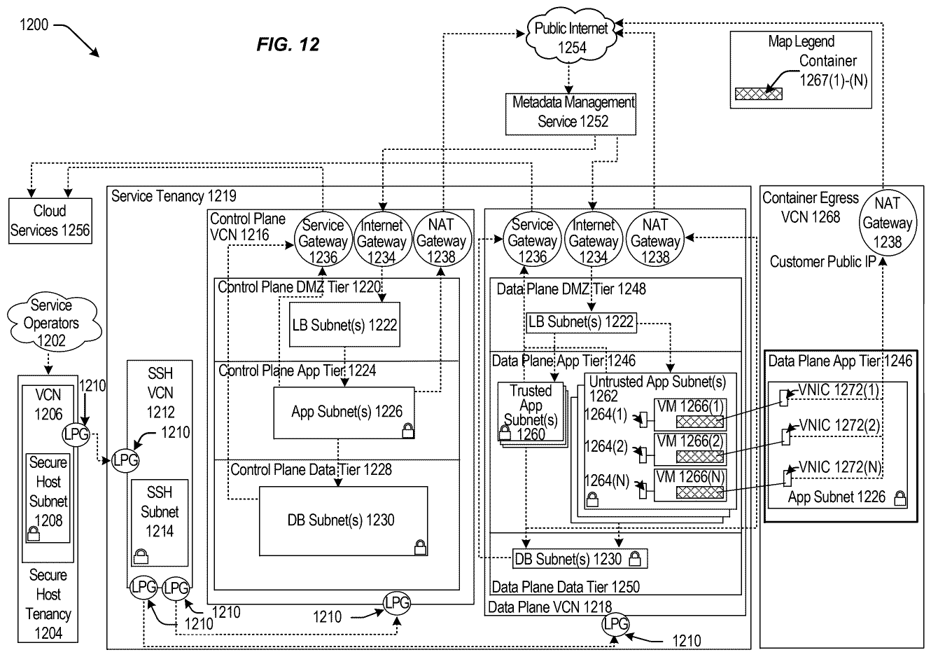

FIG. 12 is a block diagram 1200 illustrating another example pattern of an IaaS architecture, according to at least one embodiment. Service operators 1202 (e.g., service operators 902 of FIG. 9) can be communicatively coupled to a secure host tenancy 1204 (e.g., the secure host tenancy 904 of FIG. 9) that can include a virtual cloud network (VCN) 1206 (e.g., the VCN 906 of FIG. 9) and a secure host subnet 1208 (e.g., the secure host subnet 908 of FIG. 9). The VCN 1206 can include an LPG 1210 (e.g., the LPG 910 of FIG. 9) that can be communicatively coupled to an SSH VCN 1212 (e.g., the SSH VCN 912 of FIG. 9) via an LPG 1210 contained in the SSH VCN 1212. The SSH VCN 1212 can include an SSH subnet 1214 (e.g., the SSH subnet 914 of FIG. 9), and the SSH VCN 1212 can be communicatively coupled to a control plane VCN 1216 (e.g., the control plane VCN 916 of FIG. 9) via an LPG 1210 contained in the control plane VCN 1216 and to a data plane VCN 1218 (e.g., the data plane 918 of FIG. 9) via an LPG 1210 contained in the data plane VCN 1218. The control plane VCN 1216 and the data plane VCN 1218 can be contained in a service tenancy 1219 (e.g., the service tenancy 919 of FIG. 9).

The control plane VCN 1216 can include a control plane DMZ tier 1220 (e.g., the control plane DMZ tier 920 of FIG. 9) that can include LB subnet(s) 1222 (e.g., LB subnet(s) 922 of FIG. 9), a control plane app tier 1224 (e.g., the control plane app tier 924 of FIG. 9) that can include app subnet(s) 1226 (e.g., app subnet(s) 926 of FIG. 9), a control plane data tier 1228 (e.g., the control plane data tier 928 of FIG. 9) that can include DB subnet(s) 1230 (e.g., DB subnet(s) 1130 of FIG. 11). The LB subnet(s) 1222 contained in the control plane DMZ tier 1220 can be communicatively coupled to the app subnet(s) 1226 contained in the control plane app tier 1224 and to an Internet gateway 1234 (e.g., the Internet gateway 934 of FIG. 9) that can be contained in the control plane VCN 1216, and the app subnet(s) 1226 can be communicatively coupled to the DB subnet(s) 1230 contained in the control plane data tier 1228 and to a service gateway 1236 (e.g., the service gateway of FIG. 9) and a network address translation (NAT) gateway 1238 (e.g., the NAT gateway 938 of FIG. 9). The control plane VCN 1216 can include the service gateway 1236 and the NAT gateway 1238.

The data plane VCN 1218 can include a data plane app tier 1246 (e.g., the data plane app tier 946 of FIG. 9), a data plane DMZ tier 1248 (e.g., the data plane DMZ tier 948 of FIG. 9), and a data plane data tier 1250 (e.g., the data plane data tier 950 of FIG. 9). The data plane DMZ tier 1248 can include LB subnet(s) 1222 that can be communicatively coupled to trusted app subnet(s) 1260 (e.g., trusted app subnet(s) 1160 of FIG. 11) and untrusted app subnet(s) 1262 (e.g., untrusted app subnet(s) 1162 of FIG. 11) of the data plane app tier 1246 and the Internet gateway 1234 contained in the data plane VCN 1218. The trusted app subnet(s) 1260 can be communicatively coupled to the service gateway 1236 contained in the data plane VCN 1218, the NAT gateway 1238 contained in the data plane VCN 1218, and DB subnet(s) 1230 contained in the data plane data tier 1250. The untrusted app subnet(s) 1262 can be communicatively coupled to the service gateway 1236 contained in the data plane VCN 1218 and DB subnet(s) 1230 contained in the data plane data tier 1250. The data plane data tier 1250 can include DB subnet(s) 1230 that can be communicatively coupled to the service gateway 1236 contained in the data plane VCN 1218.

The untrusted app subnet(s) 1262 can include primary VNICs 1264(1)-(N) that can be communicatively coupled to tenant virtual machines (VMs) 1266(1)-(N) residing within the untrusted app subnet(s) 1262. Each tenant VM 1266(1)-(N) can run code in a respective container 1267(1)-(N), and be communicatively coupled to an app subnet 1226 that can be contained in a data plane app tier 1246 that can be contained in a container egress VCN 1268. Respective secondary VNICs 1272(1)-(N) can facilitate communication between the untrusted app subnet(s) 1262 contained in the data plane VCN 1218 and the app subnet contained in the container egress VCN 1268. The container egress VCN can include a NAT gateway 1238 that can be communicatively coupled to public Internet 1254 (e.g., public Internet 954 of FIG. 9).

The Internet gateway 1234 contained in the control plane VCN 1216 and contained in the data plane VCN 1218 can be communicatively coupled to a metadata management service 1252 (e.g., the metadata management system 952 of FIG. 9) that can be communicatively coupled to public Internet 1254. Public Internet 1254 can be communicatively coupled to the NAT gateway 1238 contained in the control plane VCN 1216 and contained in the data plane VCN 1218. The service gateway 1236 contained in the control plane VCN 1216 and contained in the data plane VCN 1218 can be communicatively coupled to cloud services 1256.

In some examples, the pattern illustrated by the architecture of block diagram 1200 of FIG. 12 may be considered an exception to the pattern illustrated by the architecture of block diagram 1100 of FIG. 11 and may be desirable for a customer of the IaaS provider if the IaaS provider cannot directly communicate with the customer (e.g., a disconnected region). The respective containers 1267(1)-(N) that are contained in the VMs 1266(1)-(N) for each customer can be accessed in real-time by the customer. The containers 1267(1)-(N) may be configured to make calls to respective secondary VNICs 127(1)-(N) contained in app subnet(s) 1226 of the data plane app tier 1246 that can be contained in the container egress VCN 1268. The secondary VNICs 1272(1)-(N) can transmit the calls to the NAT gateway 1238 that may transmit the calls to public Internet 1254. In this example, the containers 1267(1)-(N) that can be accessed in real-time by the customer can be isolated from the control plane VCN 1216 and can be isolated from other entities contained in the data plane VCN 1218. The containers 1267(1)-(N) may also be isolated from resources from other customers.

In other examples, the customer can use the containers 1267(1)-(N) to call cloud services 1256. In this example, the customer may run code in the containers 1267(1)-(N) that requests a service from cloud services 1256. The containers 1267(1)-(N) can transmit this request to the secondary VNICs 1272(1)-(N) that can transmit the request to the NAT gateway that can transmit the request to public Internet 1254. Public Internet 1254 can transmit the request to LB subnet(s) 1222 contained in the control plane VCN 1216 via the Internet gateway 1234. In response to determining the request is valid, the LB subnet(s) can transmit the request to app subnet(s) 1226 that can transmit the request to cloud services 1256 via the service gateway 1236.

It should be appreciated that IaaS architectures 900, 1000, 1100, 1200 depicted in the figures may have other components than those depicted. Further, the embodiments shown in the figures are only some examples of a cloud infrastructure system that may incorporate an embodiment of the disclosure. In some other embodiments, the IaaS systems may have more or fewer components than shown in the figures, may combine two or more components, or may have a different configuration or arrangement of components.

In certain embodiments, the IaaS systems described herein may include a suite of applications, middleware, and database service offerings that are delivered to a customer in a self-service, subscription-based, elastically scalable, reliable, highly available, and secure manner. An example of such an IaaS system is the Oracle Cloud Infrastructure (OCI) provided by the present assignee.

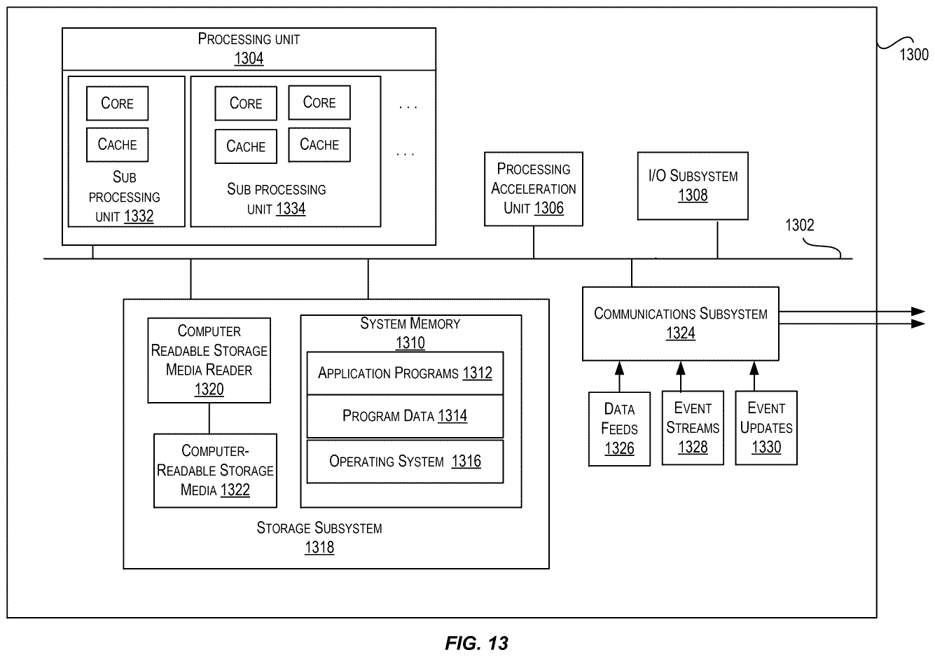

FIG. 13 illustrates an example computer system 1300, in which various embodiments may be implemented. The system 1300 may be used to implement any of the computer systems described above. As shown in the figure, computer system 1300 includes a processing unit 1304 that communicates with a number of peripheral subsystems via a bus subsystem 1302. These peripheral subsystems may include a processing acceleration unit 1306, an I/O subsystem 1308, a storage subsystem 1318 and a communications subsystem 1324. Storage subsystem 1318 includes tangible computer-readable storage media 1322 and a system memory 1310.

Bus subsystem 1302 provides a mechanism for letting the various components and subsystems of computer system 1300 communicate with each other as intended. Although bus subsystem 1302 is shown schematically as a single bus, alternative embodiments of the bus subsystem may utilize multiple buses. Bus subsystem 1302 may be any of several types of bus structures including a memory bus or memory controller, a peripheral bus, and a local bus using any of a variety of bus architectures. For example, such architectures may include an Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, which can be implemented as a Mezzanine bus manufactured to the IEEE P1386.1 standard.

Processing unit 1304, which can be implemented as one or more integrated circuits (e.g., a conventional microprocessor or microcontroller), controls the operation of computer system 1300. One or more processors may be included in processing unit 1304. These processors may include single core or multicore processors. In certain embodiments, processing unit 1304 may be implemented as one or more independent processing units 1332 and/or 1334 with single or multicore processors included in each processing unit. In other embodiments, processing unit 1304 may also be implemented as a quad-core processing unit formed by integrating two dual-core processors into a single chip.

In various embodiments, processing unit 1304 can execute a variety of programs in response to program code and can maintain multiple concurrently executing programs or processes. At any given time, some or all of the program code to be executed can be resident in processor(s) 1304 and/or in storage subsystem 1318. Through suitable programming, processor(s) 1304 can provide various functionalities described above. Computer system 1300 may additionally include a processing acceleration unit 1306, which can include a digital signal processor (DSP), a special-purpose processor, and/or the like.

I/O subsystem 1308 may include user interface input devices and user interface output devices. User interface input devices may include a keyboard, pointing devices such as a mouse or trackball, a touchpad or touch screen incorporated into a display, a scroll wheel, a click wheel, a dial, a button, a switch, a keypad, audio input devices with voice command recognition systems, microphones, and other types of input devices. User interface input devices may include, for example, motion sensing and/or gesture recognition devices such as the Microsoft Kinect® motion sensor that enables users to control and interact with an input device, such as the Microsoft Xbox® 560 game controller, through a natural user interface using gestures and spoken commands. User interface input devices may also include eye gesture recognition devices such as the Google Glass® blink detector that detects eye activity (e.g., ‘blinking’ while taking pictures and/or making a menu selection) from users and transforms the eye gestures as input into an input device (e.g., Google Glass®). Additionally, user interface input devices may include voice recognition sensing devices that enable users to interact with voice recognition systems (e.g., Siri® navigator), through voice commands.

User interface input devices may also include, without limitation, three dimensional (3D) mice, joysticks or pointing sticks, gamepads and graphic tablets, and audio/visual devices such as speakers, digital cameras, digital camcorders, portable media players, webcams, image scanners, fingerprint scanners, barcode reader 5D scanners, 5D printers, laser rangefinders, and eye gaze tracking devices. Additionally, user interface input devices may include, for example, medical imaging input devices such as computed tomography, magnetic resonance imaging, position emission tomography, medical ultrasonography devices. User interface input devices may also include, for example, audio input devices such as MIDI keyboards, digital musical instruments and the like.

User interface output devices may include a display subsystem, indicator lights, or non-visual displays such as audio output devices, etc. The display subsystem may be a cathode ray tube (CRT), a flat-panel device, such as that using a liquid crystal display (LCD) or plasma display, a projection device, a touch screen, and the like. In general, use of the term “output device” is intended to include all possible types of devices and mechanisms for outputting information from computer system 1300 to a user or other computer. For example, user interface output devices may include, without limitation, a variety of display devices that visually convey text, graphics and audio/video information such as monitors, printers, speakers, headphones, automotive navigation systems, plotters, voice output devices, and modems.

Computer system 1300 may comprise a storage subsystem 1318 that provides a tangible non-transitory computer-readable storage medium for storing software and data constructs that provide the functionality of the embodiments described in this disclosure. The software can include programs, code modules, instructions, scripts, etc., that when executed by one or more cores or processors of processing unit 1304 provide the functionality described above. Storage subsystem 1318 may also provide a repository for storing data used in accordance with the present disclosure.

As depicted in the example in FIG. 13, storage subsystem 1318 can include various components including a system memory 1310, computer-readable storage media 1322, and a computer readable storage media reader 1320. System memory 1310 may store program instructions that are loadable and executable by processing unit 1304. System memory 1310 may also store data that is used during the execution of the instructions and/or data that is generated during the execution of the program instructions. Various different kinds of programs may be loaded into system memory 1310 including but not limited to client applications, Web browsers, mid-tier applications, relational database management systems (RDBMS), virtual machines, containers, etc.

System memory 1310 may also store an operating system 1316. Examples of operating system 1316 may include various versions of Microsoft Windows®, Apple Macintosh®, and/or Linux operating systems, a variety of commercially-available UNIX® or UNIX-like operating systems (including without limitation the variety of GNU/Linux operating systems, the Google Chrome® OS, and the like) and/or mobile operating systems such as iOS, Windows® Phone, Android® OS, BlackBerry® OS, and Palm® OS operating systems. In certain implementations where computer system 1300 executes one or more virtual machines, the virtual machines along with their guest operating systems (GOSs) may be loaded into system memory 1310 and executed by one or more processors or cores of processing unit 1304.

System memory 1310 can come in different configurations depending upon the type of computer system 1300. For example, system memory 1310 may be volatile memory (such as random access memory (RAM)) and/or non-volatile memory (such as read-only memory (ROM), flash memory, etc.) Different types of RAM configurations may be provided including a static random access memory (SRAM), a dynamic random access memory (DRAM), and others. In some implementations, system memory 1310 may include a basic input/output system (BIOS) containing basic routines that help to transfer information between elements within computer system 1300, such as during start-up.

Computer-readable storage media 1322 may represent remote, local, fixed, and/or removable storage devices plus storage media for temporarily and/or more permanently containing, storing, computer-readable information for use by computer system 1300 including instructions executable by processing unit 1304 of computer system 1300.

Computer-readable storage media 1322 can include any appropriate media known or used in the art, including storage media and communication media, such as but not limited to, volatile and non-volatile, removable and non-removable media implemented in any method or technology for storage and/or transmission of information. This can include tangible computer-readable storage media such as RAM, ROM, electronically erasable programmable ROM (EEPROM), flash memory or other memory technology, CD-ROM, digital versatile disk (DVD), or other optical storage, magnetic cassettes, magnetic tape, magnetic disk storage or other magnetic storage devices, or other tangible computer readable media.

By way of example, computer-readable storage media 1322 may include a hard disk drive that reads from or writes to non-removable, nonvolatile magnetic media, a magnetic disk drive that reads from or writes to a removable, nonvolatile magnetic disk, and an optical disk drive that reads from or writes to a removable, nonvolatile optical disk such as a CD ROM, DVD, and Blu-Ray® disk, or other optical media. Computer-readable storage media 1322 may include, but is not limited to, Zip® drives, flash memory cards, universal serial bus (USB) flash drives, secure digital (SD) cards, DVD disks, digital video tape, and the like. Computer-readable storage media 1322 may also include, solid-state drives (SSD) based on non-volatile memory such as flash-memory based SSDs, enterprise flash drives, solid state ROM, and the like, SSDs based on volatile memory such as solid state RAM, dynamic RAM, static RAM, DRAM-based SSDs, magnetoresistive RAM (MRAM) SSDs, and hybrid SSDs that use a combination of DRAM and flash memory based SSDs. The disk drives and their associated computer-readable media may provide non-volatile storage of computer-readable instructions, data structures, program modules, and other data for computer system 1300.

Machine-readable instructions executable by one or more processors or cores of processing unit 1304 may be stored on a non-transitory computer-readable storage medium. A non-transitory computer-readable storage medium can include physically tangible memory or storage devices that include volatile memory storage devices and/or non-volatile storage devices. Examples of non-transitory computer-readable storage medium include magnetic storage media (e.g., disk or tapes), optical storage media (e.g., DVDs, CDs), various types of RAM, ROM, or flash memory, hard drives, floppy drives, detachable memory drives (e.g., USB drives), or other type of storage device.

Communications subsystem 1324 provides an interface to other computer systems and networks. Communications subsystem 1324 serves as an interface for receiving data from and transmitting data to other systems from computer system 1300. For example, communications subsystem 1324 may enable computer system 1300 to connect to one or more devices via the Internet. In some embodiments communications subsystem 1324 can include radio frequency (RF) transceiver components for accessing wireless voice and/or data networks (e.g., using cellular telephone technology, advanced data network technology, such as 5G, 4G or EDGE (enhanced data rates for global evolution), WiFi (IEEE 802.11 family standards, or other mobile communication technologies, or any combination thereof)), global positioning system (GPS) receiver components, and/or other components. In some embodiments communications subsystem 1324 can provide wired network connectivity (e.g., Ethernet) in addition to or instead of a wireless interface.

In some embodiments, communications subsystem 1324 may also receive input communication in the form of structured and/or unstructured data feeds 1326, event streams 1328, event updates 1330, and the like on behalf of one or more users who may use computer system 1300.

By way of example, communications subsystem 1324 may be configured to receive data feeds 1326 in real-time from users of social networks and/or other communication services such as Twitter® feeds, Facebook® updates, web feeds such as Rich Site Summary (RSS) feeds, and/or real-time updates from one or more third party information sources.

Additionally, communications subsystem 1324 may also be configured to receive data in the form of continuous data streams, which may include event streams 1328 of real-time events and/or event updates 1330, that may be continuous or unbounded in nature with no explicit end. Examples of applications that generate continuous data may include, for example, sensor data applications, financial tickers, network performance measuring tools (e.g., network monitoring and traffic management applications), clickstream analysis tools, automobile traffic monitoring, and the like.

Communications subsystem 1324 may also be configured to output the structured and/or unstructured data feeds 1326, event streams 1328, event updates 1330, and the like to one or more databases that may be in communication with one or more streaming data source computers coupled to computer system 1300.

Computer system 1300 can be one of various types, including a handheld portable device (e.g., an iPhone® cellular phone, an iPad® computing tablet, a PDA), a wearable device (e.g., a Google Glass® head mounted display), a PC, a workstation, a mainframe, a kiosk, a server rack, or any other data processing system.

Due to the ever-changing nature of computers and networks, the description of computer system 1300 depicted in the figure is intended only as a specific example. Many other configurations having more or fewer components than the system depicted in the figure are possible. For example, customized hardware might also be used and/or particular elements might be implemented in hardware, firmware, software (including applets), or a combination. Further, connection to other computing devices, such as network input/output devices, may be employed. Based on the disclosure and teachings provided herein, a person of ordinary skill in the art will appreciate other ways and/or methods to implement the various embodiments.

Although specific embodiments have been described, various modifications, alterations, alternative constructions, and equivalents are also encompassed within the scope of the disclosure. Embodiments are not restricted to operation within certain specific data processing environments, but are free to operate within a plurality of data processing environments. Additionally, although embodiments have been described using a particular series of transactions and steps, it should be apparent to those skilled in the art that the scope of the present disclosure is not limited to the described series of transactions and steps. Various features and aspects of the above-described embodiments may be used individually or jointly.

Further, while embodiments have been described using a particular combination of hardware and software, it should be recognized that other combinations of hardware and software are also within the scope of the present disclosure. Embodiments may be implemented only in hardware, or only in software, or using combinations thereof. The various processes described herein can be implemented on the same processor or different processors in any combination. Accordingly, where components or services are described as being configured to perform certain operations, such configuration can be accomplished, e.g., by designing electronic circuits to perform the operation, by programming programmable electronic circuits (such as microprocessors) to perform the operation, or any combination thereof. Processes can communicate using a variety of techniques including but not limited to conventional techniques for inter process communication, and different pairs of processes may use different techniques, or the same pair of processes may use different techniques at different times.

The specification and drawings are, accordingly, to be regarded in an illustrative rather than a restrictive sense. It will, however, be evident that additions, subtractions, deletions, and other modifications and changes may be made thereunto without departing from the broader spirit and scope as set forth in the claims. Thus, although specific disclosure embodiments have been described, these are not intended to be limiting. Various modifications and equivalents are within the scope of the following claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known for carrying out the disclosure. Variations of those preferred embodiments may become apparent to those of ordinary skill in the art upon reading the foregoing description. Those of ordinary skill should be able to employ such variations as appropriate and the disclosure may be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein.

All references, including publications, patent applications, and patents, cited herein are hereby incorporated by reference to the same extent as if each reference were individually and specifically indicated to be incorporated by reference and were set forth in its entirety herein.

In the foregoing specification, aspects of the disclosure are described with reference to specific embodiments thereof, but those skilled in the art will recognize that the disclosure is not limited thereto. Various features and aspects of the above-described disclosure may be used individually or jointly. Further, embodiments can be utilized in any number of environments and applications beyond those described herein without departing from the broader spirit and scope of the specification. The specification and drawings are, accordingly, to be regarded as illustrative rather than restrictive.

What is claimed is:

1. A computer-implemented method comprising:
receiving, at a data storage system comprising a plurality of data stores, a query, wherein:
the query is associated with one or more constraints,
the one or more constraints comprise (i) a freshness constraint, (ii) a feasibility constraint, (iii) an execution time constraint, or (iv) any combination thereof, and
the query is in a first programming language;
identifying, from a set of intents, an intent of the query based on one or more key terms within the query;
performing a dry run of the query on at least a subset of the plurality of data stores to evaluate, for each data store of at least the subset, whether the data store satisfies the one or more constraints based on the intent and data store metadata, wherein the data store metadata comprises data store information associated with each data store of at least the subset;
selecting, based on the dry run, an optimal data store from at least the subset of the plurality of data stores;
generating a query result for the query, wherein generating the query result comprises:
determining whether the query in the first programming language can be executed on the optimal data store,
in response to determining the query in the first programming language can be executed on the optimal data store, executing the query on the optimal data store to obtain the query result, and
in response to determining the query in the first programming language cannot be executed on the optimal data store: converting the query to a second query in a second programming language that can be executed on the optimal data store, and executing the second query on the optimal data store to obtain the query result; and
providing the query result.

2. The computer-implemented method of claim 1, wherein the data store information is associated with (i) a data model of the data store, (ii) a schema of the data store, (iii) a set of features of the data store, or (iv) any combination thereof.

3. The computer-implemented method of claim 1, wherein the set of intents comprises (i) point query, (ii) filter query, (iii) join query, (iv) aggregate query, (v) subquery, or (vi) any combination thereof.

4. The computer-implemented method of claim 1, wherein the one or more constraints comprise the freshness constraint, and wherein performing the dry run comprises, for each data store of at least the subset:
computing a watermark indicating a freshness of data within the data store based on the data store metadata; and
determining whether the data store satisfies the freshness constraint based on a comparison of the watermark and an expected freshness indicated by the freshness constraint.

5. The computer-implemented method of claim 1, wherein:
the one or more constraints comprise the feasibility constraint;
performing the dry run comprises, for each data store of at least the subset, determining whether the query can be executed on the data store based on the intent and one or more features of the data store; and
the one or more features are determined based on the data store metadata.

6. The computer-implemented method of claim 1, wherein performing the dry run comprises, for each data store of at least the subset:
determining a constraint satisfaction score for each constraint of the one or more constraints based on the intent and the data store metadata;
computing a weighted sum of the constraint satisfaction scores;
determining whether the weighted sum matches or exceeds a constraint satisfaction threshold;
in response to determining the weighted sum matches or exceeds the constraint satisfaction threshold, determining the data store satisfies the one or more constraints; and
in response to determining the weighted sum does not match or exceed the constraint satisfaction threshold, determining the data store does not satisfy the one or more constraints.

7. The computer-implemented method of claim 1, wherein the one or more constraints are expressed in a constraint definition language (CDL), and wherein the expression of the one or more constraints in the CDL indicates a logical precedence of the one or more constraints.

8. The computer-implemented method of claim 1, further comprising:
collecting execution metadata associated with the execution of the query or the execution of the second query, the execution metadata comprising at least one of (i) query response time, (ii) freshness level, (iii) feasibility status, (iv) user satisfaction, or (v) any combination thereof;
receiving a subsequent query associated with at least one of the one or more constraints; and
selecting the optimal data store based on the execution metadata and historical query execution data.

9. The computer-implemented method of claim 1, wherein converting the query comprises:
providing, to a generative model, a prompt comprising the query and one or more instructions to translate the query from the first programming language to the second programming language; and
receiving, from the generative model, the second query corresponding to the second programming language, wherein the generative model generates the second query based on the prompt, wherein the second programming language is a programming language that can be executed on the optimal data store.

10. The computer-implemented method of claim 1, wherein selecting the optimal data store comprises applying a machine learning decision model trained on historical query execution patterns.

11. A system comprising:
a data storage system comprising a plurality of data stores;
one or more processors; and
one or more computer-readable media storing instructions which, when executed by the one or more processors, cause the system to perform operations comprising:
receiving, at the data storage system, a query, wherein:
the query is associated with one or more constraints,
the one or more constraints comprise (i) a freshness constraint, (ii) a feasibility constraint, (iii) an execution time constraint, or (iv) any combination thereof, and
the query is in a first programming language;
identifying, from a set of intents, an intent of the query based on one or more key terms within the query;
performing a dry run of the query on at least a subset of the plurality of data stores to evaluate, for each data store of at least the subset, whether the data store satisfies the one or more constraints based on the intent and data store metadata, wherein the data store metadata comprises data store information associated with each data store of at least the subset;
selecting, based on the dry run, an optimal data store from at least the subset of the plurality of data stores;
generating a query result for the query, wherein generating the query result comprises:
determining whether the query in the first programming language can be executed on the optimal data store,
in response to determining the query in the first programming language can be executed on the optimal data store, executing the query on the optimal data store to obtain the query result, and
in response to determining the query in the first programming language cannot be executed on the optimal data store:
converting the query to a second query in a second programming language that can be executed on the optimal data store, and
executing the second query on the optimal data store to obtain the query result; and
providing the query result to a user or an entity.

12. The system of claim 11, wherein the one or more constraints comprise the freshness constraint, and wherein performing the dry run comprises, for each data store of at least the subset:
computing a watermark indicating a freshness of data within the data store based on the data store metadata; and
determining whether the data store satisfies the freshness constraint based on a comparison of the watermark and an expected freshness indicated by the freshness constraint.

13. The system of claim 11, wherein:
the one or more constraints comprise the feasibility constraint;
performing the dry run comprises, for each data store of at least the subset, determining whether the query can be executed on the data store based on the intent and one or more features of the data store; and
the one or more features are determined based on the data store metadata.

14. The system of claim 11, wherein performing the dry run comprises, for each data store of at least the subset:
determining a constraint satisfaction score for each constraint of the one or more constraints based on the intent and the data store metadata;
computing a weighted sum of the constraint satisfaction scores;
determining whether the weighted sum matches or exceeds a constraint satisfaction threshold;
in response to determining the weighted sum matches or exceeds the constraint satisfaction threshold, determining the data store satisfies the one or more constraints; and
in response to determining the weighted sum does not match or exceed the constraint satisfaction threshold, determining the data store does not satisfy the one or more constraints.

15. The system of claim 11, wherein the one or more constraints are expressed in a constraint definition language (CDL), and wherein the expression of the one or more constraints in the CDL indicates a logical precedence of the one or more constraints.

16. The system of claim 11, wherein the operations further comprise:
collecting execution metadata associated with the execution of the query or the execution of the second query, the execution metadata comprising at least one of (i) query response time, (ii) freshness level, (iii) feasibility status, (iv) user satisfaction, or (v) any combination thereof;
receiving a subsequent query associated with at least one of the one or more constraints; and
selecting the optimal data store based on the execution metadata and historical query execution data.

17. One or more non-transitory computer-readable media storing instructions which, when executed by one or more processors, cause the one or more processors to perform operations comprising:
receiving, at a data storage system comprising a plurality of data stores, a query, wherein the query is associated with one or more constraints, and wherein the query is in a first programming language;
identifying, from a set of intents, an intent of the query based on one or more key terms within the query;
performing a dry run of the query on at least a subset of the plurality of data stores to evaluate, for each data store of at least the subset, whether the data store satisfies the one or more constraints based on the intent and data store metadata, wherein the data store metadata comprises data store information associated with each data store of at least the subset;
selecting, based on the dry run, an optimal data store from at least the subset of the plurality of data stores;
generating a query result for the query, wherein generating the query result comprises:
determining whether the query in the first programming language can be executed on the optimal data store,
in response to determining the query in the first programming language can be executed on the optimal data store, executing the query on the optimal data store to obtain the query result, and
in response to determining the query in the first programming language cannot be executed on the optimal data store:
converting the query to a second query in a second programming language that can be executed on the optimal data store, and
executing the second query on the optimal data store to obtain the query result; and
providing the query result to a user or an entity.

18. The one or more non-transitory computer-readable media of claim 17, wherein the one or more constraints comprise the freshness constraint, and wherein performing the dry run comprises, for each data store of at least the subset:
computing a watermark indicating a freshness of data within the data store based on the data store metadata; and
determining whether the data store satisfies the freshness constraint based on a comparison of the watermark and an expected freshness indicated by the freshness constraint.

19. The one or more non-transitory computer-readable media claim 17, wherein:
the one or more constraints comprise the feasibility constraint;
performing the dry run comprises, for each data store of at least the subset, determining whether the query can be executed on the data store based on the intent and one or more features of the data store; and
the one or more features are determined based on the data store metadata.

20. The one or more non-transitory computer-readable media claim 17, wherein performing the dry run comprises, for each data store of at least the subset:
determining a constraint satisfaction score for each constraint of the one or more constraints based on the intent and the data store metadata;
computing a weighted sum of the constraint satisfaction scores;
determining whether the weighted sum matches or exceeds a constraint satisfaction threshold;
in response to determining the weighted sum matches or exceeds the constraint satisfaction threshold, determining the data store satisfies the one or more constraints; and
in response to determining the weighted sum does not match or exceed the constraint satisfaction threshold, determining the data store does not satisfy the one or more constraints.
